(19) United States
(12) Patent Application Publication
(10) Pub. No.: US 2004/0185094 A1
(43) Pub. Date: Sep. 23, 2004

(54) Pharmaceutical

(76) Inventors: Graham Nigel Maw, Sandwich (GB); Christopher Peter Wayman, Sandwich (GB)

Correspondence Address: Gregg C. Benson, Pfizer Inc., Patent Department, MS 4159, Eastern Point Road, Groton, CT, 06340, US

(21) Appl. No.: 09/939,093

(22) Filed: Aug. 24, 2001

Related U.S. Application Data
(60) Provisional application No. 60/238,206, filed on Oct. 5, 2000.

(30) Foreign Application Priority Data
Sep. 1, 2000 (GB) 0021487.4

(51) Int. Cl. A61K 9/20; A61K 31/517

(52) U.S. Cl. 424/464; 514/252.16

(57) ABSTRACT

A method of treating an individual is described. The method comprise delivering to the individual an agent that is capable of modulating an intermediate conductance calcium-activated potassium (IK_{Ca}) channel in the sexual genitalia of the individual; wherein the modulation of the IK_{Ca} channel by the agent is capable of mediating a relaxation of corpus cavernosal smooth muscle tone. The agent may be admixed with a pharmaceutically acceptable carrier, diluent or exicipient.

### FIELD OF THE INVENTION

The present invention relates to a pharmaceutical that is useful for the prevention and/or treatment of sexual dysfunction (SD), in particular male erectile dysfunction (MED).

The present invention also relates to a method of prevention and/or treatment of SD, in particular MED.

The present invention also relates to assays to screen for compounds useful in the treatment of SD, in particular MED.

For convenience, a list of abbreviations that are used in the following text is presented before the Claims section.

### BACKGROUND TO THE INVENTION

Sexual dysfunction (SD) is a significant clinical problem which can affect both males and females. The causes of SD may be both organic and physiological. Organic aspects of SD are typically caused by underlying vascular diseases, such as those associated with hypertension or diabetes mellitus, by prescription medication and/or by psychiatric disease such as depression. Physiological factors include fear, performance anxiety and interpersonal conflict. SD impairs sexual performance, diminishes self-esteem and disrupts personal relationships. In the clinic, SD disorders have been divided into female sexual dysfunction (FSD) disorders and male sexual dysfunction (MSD) disorders. FSD is best defined as the difficulty or inability of a woman to find satisfaction in sexual expression. FSD is a collective term for several diverse female sexual disorders (Leiblum, 1998, Berman et al 1999). Male sexual dysfunction (MSD) is generally associated with erectile dysfunction, also known as male erectile dysfunction (MED).

Male erectile dysfunction (MED) is defined as:

“the inability to achieve and/or maintain a penile erection for satisfactory sexual performance” (NIH Consensus Development Panel on Impotence, 1993)”

It has been estimated that the prevalence of erectile dysfunction (ED) of all degrees (minimal, moderate and complete impotence) is 52% in men 40 to 70 years old, with higher rates in those older than 70 (Melman et al 1999). The condition has a significant negative impact on the quality of life of the patient and their partner, often resulting in increased anxiety and tension which leads to depression and low self esteem. Whereas 2 decades ago, MED was primarily considered to be a psychological disorder (Benet et al 1994), it is now known that for the majority of patients there is an underlying organic cause. As a result, much progress has been made in identifying the mechanism of normal penile erection and the pathophysiology (pathophysiologies) of MED.

Penile erection is a haemodynamic event which is dependent upon the balance of contraction and relaxation of the corpus cavernosal smooth muscle and arterioles of the penis (Lerner et al 1993). Corporal smooth muscle contraction is modulated by sympathetic noradrenergic innervation via activation of postsynaptic α₁ adrenoceptors. Relaxation of the cavernosal smooth muscle leads to an increased blood flow into the trabecular spaces of the corpus cavernosa, causing them to expand against the surrounding tunica and compress the draining veins. This produces a vast elevation in blood pressure which results in an erection (Naylor, 1998).

The changes that occur during the erectile process are complex and require a high degree of coordinated control involving the peripheral and central nervous systems, and the endocrine system (Naylor, 1998). However, the process of smooth muscle relaxation is mediated primarily by non-adrenergic, non-cholinergic (NANC) neurotransmission. The main relaxing factor responsible for mediating this relaxation is nitric oxide (NO), which is synthesised from L-arginine by nitric oxide synthase (NOS) (Taub et al 1993; Chuang et al 1998). During erection, NO is released from neurones and the endothelium and binds to and activates soluble guanylate cyclase (sGC) located in the smooth muscle cells and endothelium, leading to an elevation in intracellular cGMP levels. This rise in cGMP leads to a relaxation of the corpus cavernosum.

MED mainly arises from an inability of NO to effectively relax corpus cavernosum smooth muscle. It is therefore possible to treat MED by potentiating or facilitating NO signalling leading to an elevation in intracellular cGMP levels. In this respect, sildenafil citrate (also known as Viagra™) has recently been developed by Pfizer as the first oral drug treatment for MED.

Sildenafil acts by inhibiting cGMP breakdown in the corpus cavernosum by selectively inhibiting phosphodiesterase 5 (PDE5), thereby preventing the hydrolysis of cGMP to 5′GMP (Boolel et al 1996; Jeremy et al 1997). Sildenafil currently represents the most preferred therapy on the market as other injectable vasoactive drugs commonly show high efficacy, side effects such as penile pain, fibrosis and priapism (that is, erections of inappropriate overlong duration) are common. Currently, all other available therapies are invasive and include vacuum constriction devices, vasoactive drug injection and penile prostheses implantation (Montague et al 1996).

Thus, it is desirable to find new ways of treating SD.

### SUMMARY ASPECTS

The present invention is based on the novel finding that SD may be associated with intermediate-conductance calcium-activated potassium (IK_{Ca}) channel activity. This is the first report documenting a role for IK_{Ca} channels in the regulation of corpus cavernosal smooth muscle tone. More importantly, this is the first report demonstrating a role for IK_{Ca} channels in mediating NO-induced relaxations of corpus cavernosal smooth muscle tone. These findings have important implications for the treatment of MED.

In this regard, the IK_{Ca} channels may be used as a therapeutic target to mediate either the direct relaxation of corpus cavernosal smooth muscle or to potentiate NO-induced relaxations of corpus cavernosal smooth muscle. Prophylactic and/or therapeutic treatment of an individual may be achieved using an agent capable of modulating IK_{Ca} channel activity such that, for example, IK_{Ca} channel opening is increased and/or the duration of IK_{Ca} channel opening is increased and/or the probability of IK_{Ca} channel opening is increased and/or the open time probability of the IK_{Ca} channel is increased. The agent may directly or indirectly enhance channel opening by, for example, increasing the calcium sensitivity of the IK_{Ca} channel. This effect of direct channel opening or increase in calcium sensitivity causes the IK_{Ca} channel to open which promotes the relaxation of smooth muscle tone in the corpus cavernosum. As well as increasing the calcium sensitivity of the IK_{Ca} channel, the agent may also increase the sensitivity of the IK_{Ca} channel to nitric oxide (NO). NO may then increase calcium sensitivity either directly or indirectly via the PKG/cGMP pathway. Any deficiency in NO signalling associated with, for example MED, may thus be overcome by using an IK_{Ca} channel modulator/activator/opener to enhance nitrergic relaxations of corpus cavernosal smooth muscle.

As indicated, the present invention is the first report that SD may be associated with intermediate-conductance calcium-activated potassium (IK_{Ca}) channel activity. This is in direct contrast to the prior art teachings on the possible involvement of potassium channels in SD. In this respect, the prior art concerns the opening of large conductance calcium-activated potassium channels (BK channels). By way of example, reference may be made to WO-A-99/38853 and WO-A-99/09983. Reference may also be made to Moreland et al (2001) (The Journal of Pharmacology and Experimental Therapeutics vol 296 (no. 2) pages 225-234) and Moreland et al (2000) (Current Opinion in CPNS Investigational Drugs vol 2 (no. 3) pages 283-302) who report on the use of agents that target BK channels. U.S. Pat. No. 5,430,048 is silent on which potassium channel is targeted.

Hence, the role for IK_{Ca} channels in the regulation of corpus cavernosal smooth muscle tone represents a very important finding.

Thus, in one aspect, the present invention provides the use of an IK_{Ca} channel modulator for the treatment of SD.

In another aspect, the present invention provides the use of an IK_{Ca} channel modulator as a smooth muscle relaxant.

In another aspect, the present invention provides the use of an IK_{Ca} channel modulator as a urogenital smooth muscle relaxant.

### DETAILED ASPECTS OF THE INVENTION

In one aspect, the present invention relates to a pharmaceutical composition for subsequent use in the treatment of a sexual dysfunction (SD); the pharmaceutical composition comprising an agent capable of modulating the activity of an intermediate conductance calcium-activated potassium (IK_{Ca}) channel in the sexual genitalia of an individual; wherein the agent is optionally admixed with a pharmaceutically acceptable carrier, diluent or excipient.

Alternatively expressed, the present invention relates to a pharmaceutical composition for use (or when in use) in the treatment of a sexual dysfunction (SD); the pharmaceutical composition comprising an agent capable of modulating the activity of an intermediate conductance calcium-activated potassium (IK_{Ca}) channel in the sexual genitalia of an individual; wherein the agent is optionally admixed with a pharmaceutically acceptable carrier, diluent or excipient.

In another aspect, the present invention relates to the use of an agent in the preparation of a medicament for the treatment of a SD; wherein the agent is capable of modulating an IK_{Ca} channel activity in the sexual genitalia of an individual; wherein the agent is optionally admixed with a pharmaceutically acceptable carrier, diluent or excipient.

In a further aspect, the present invention relates to a method for treating an individual; the method comprising delivering to the individual an agent that is capable of modulating IK_{Ca} channel activity in the sexual genitalia of the individual; wherein the agent is optionally admixed with a pharmaceutically acceptable carrier, diluent or excipient.

In a further aspect, the present invention relates to an assay method for identifying an agent capable of modulating an IK_{Ca} channel activity; the assay method comprising: contacting the agent with the IK_{Ca} channel; measuring the IK_{Ca} channel activity; wherein an increase in the IK_{Ca} channel activity is indicative that the agent may be useful in the prevention and/or treatment of the SD.

In a further aspect, the present invention relates to a process comprising the steps of:

(a) performing the assay according to the present invention; (b) identifying one or more agents capable of modulating an IK_{Ca} channel activity; and (c) preparing a quantity of those one or more identified agents.

In a further aspect, the present invention relates to a method of preventing and/or treating a SD with an agent; wherein the agent is capable of modulating an IK_{Ca} channel activity in an in vitro assay method; wherein the in vitro assay method is the assay method according to the present invention.

In a further aspect, the present invention relates to the use of an agent in the preparation of a pharmaceutical composition for the prevention and/or treatment of a SD; wherein the agent is capable of modulating an IK_{Ca} channel activity when assayed in vitro by the assay method according to the present invention.

In a further aspect, the present invention relates to a diagnostic method wherein the method comprises: isolating a sample from the sexual genitalia of an individual; determining whether the expression and/or IK_{Ca} channel activity in the sample from the individual has an effect on the relaxation of corpus cavernosal smooth muscle tone in the sexual genitalia of the individual.

In a further aspect, the present invention relates to a diagnostic composition or kit comprising means for detecting an entity in an isolated sample from the sexual genitalia of an individual; wherein the means can be used for determining whether the expression of the IK_{Ca} channel and/or the level of IK_{Ca} channel activity in the sample from the individual has an effect on the relaxation of corpus cavernosal smooth muscle tone in the sexual genitalia of the individual.

In a further aspect, the present invention relates to an animal model useful in the identification of agents capable of preventing and/or treating a SD; said model comprising an anaesthetised animal; and including means to measure IK_{Ca} channel activity in the corpus cavernosal smooth muscle cells of said animal.

In a further aspect, the present invention relates to an assay method for identifying an agent capable of modulating an IK_{Ca} channel activity in order to prevent and/or treat a SD (preferably MED); the assay method comprising: administering an agent to the animal model according to the present invention and measuring the ionic conductance passing through IK_{Ca} channel or membrane potential in the sexual genitalia of said animal.

In a further aspect, the present invention relates to an IK_{Ca} channel for use in medicine.

In a further aspect, the present invention relates to the use of an IK_{Ca} channel in the preparation of a medicament to prevent and/or treat a SD (preferably MED). Here, the IK_{Ca} channel may be used in, for example, a manufacturing step and/or an identification preparative step and/or a modification preparative step of an agent according to the present invention.

In a further aspect, the present invention relates to an IK_{Ca} channel as a target to identify agents capable of mediating the relaxation of corpus cavernosal smooth muscle tone.

In a further aspect, the present invention relates to an IK_{Ca} channel to screen for agents capable of modulating IK_{Ca} channel activity.

In a further aspect, the present invention relates to the use of an IK_{Ca} channel in the manufacture of a medicament to prevent and/or treat a SD (preferably MED). Here, the IK_{Ca} channel is formulated into a medicament.

Other aspects of the present invention are presented in the accompanying claims and in the following description and drawings. These aspects are presented under separate section headings. However, it is to be understood that the teachings under each section are not necessarily limited to that particular section heading.

Preferable Aspects

Preferably the agent is for the treatment of a SD.

The SD may be a male SD or a female SD.

Preferably the agent is for the treatment of a SD wherein the SD is an erectile dysfunction (ED).

Preferably the agent is for the treatment of an ED such as MED.

Preferably the modulation of IK_{Ca} channel activity increases IK_{Ca} channel opening and/or increases the duration of IK_{Ca} channel opening and/or increases the probability of IK_{Ca} channel opening and/or increases the open time probability of the IK_{Ca} channel.

Preferably the agent capable of modulating IK_{Ca} channel activity is capable of mediating a relaxation of corpus cavernosal smooth muscle tone.

Preferably the agent capable of modulating IK_{Ca} channel activity enhances nitregic or nitric oxide-mediated relaxation of corpus cavernosal smooth muscle tone.

In one embodiment, preferably the agent is for oral administration.

In another embodiment, the agent may be for topical administration or intracavernosal administration.

The present invention also encompasses administration of the agent of the present invention before and/or during sexual arousal/stimulation.

This is advantageous because it provides systemic selectivity. The natural cascade only occurs at the genitalia and not the heart etc, hence it would be possible to achieve a selective effect on the genitalia.

Thus, for some aspects of the present invention it is highly desirable that there is a sexual arousal/stimulation step. We have found that this step can provide systemic selectivity.

Here, “sexual arousal/stimulation” may be one or more of a visual arousal/stimulation, a physical arousal/stimulation, an auditory arousal/stimulation or a thought arousal/stimulation.

Thus, preferably the agents of the present invention are delivered before or during sexual arousal/stimulation, particularly when those agents are for oral delivery.

Hence, for this preferred aspect, the present invention provides for the use of an agent in the manufacture of a medicament for the treatment of SD; wherein the agent is capable of modulating IK_{Ca} channel activity in an individual; and wherein said individual is sexually aroused/stimulated before or during administration of said medicament.

Preferably, said medicament is delivered orally to said individual.

In addition, for this preferred aspect, the present invention provides for a method of treating an individual; the method comprising delivering to the individual an agent that is capable of modulating IK_{Ca} channel activity; wherein the agent is in an amount to treat SD; wherein the agent is optionally admixed with a pharmaceutically acceptable carrier, diluent or excipient; and wherein said individual is sexually aroused/stimulated before or during administration of said agent.

Preferably, said agent is delivered orally to said individual.

Surprising and Unexpected Findings

The present invention demonstrates the surprising and unexpected findings that:

(a) IK_{Ca} channels are expressed in corpus cavernosal smooth muscle even though they are not expressed in vascular smooth muscle (such as heart, aorta) or skeletal muscle.

(b) The modulation of IK_{Ca} channel activity in corpus cavernosal smooth muscle induces a relaxation of smooth muscle tone which is capable of mediating an erectile response, such as penile erection.

(c) The modulation of IK_{Ca} channel activity may mediate a relaxation of corpus cavernosal smooth muscle tone by, for example, muscle hyperpolarisation and/or inhibiting calcium channel activity (such as voltage operated calcium channels) or by potentiating a NO-mediated relaxation of smooth muscle tone.

(d) The enhanced relaxant effects mediated by increased opening of IK_{Ca} activity may be mediated directly or indirectly by an increased sensitivity to intracellular calcium concentrations.

(e) The NO-mediated relaxation of corpus cavernosal smooth muscle tone may be mediated, in part, by opening of IK_{Ca} channels. NO may increase IK_{Ca} channel opening directly via an endogenous nitrergic pathway or indirectly via the PKG/cGMP mediated pathway.

(f) IK_{Ca} channel openers, such as EBIO (1-ethyl-2-benzimidazolinone), may play a role in the modulation of IK_{Ca} channel activity by enhancing a NO-induced relaxation of corpus cavernosal smooth muscle tone. Such a NO-induced relaxation of smooth muscle tone may also be observed in response to sexual arousal.

(g) An agent capable of modulating IK_{Ca} channel activity may be useful in enhancing the erectile response and may help to overcome an erectile dysfunction such as MED.

(h) IK_{Ca} channel opening enhances NO-mediated relaxation of corpus cavernosal smooth muscle tone and thus enhances the endogenous erectile process.

Advantages

The present invention is advantageous because:

(i) agents which play a role in the modulation of IK_{Ca} channel activity can provide a means for preventing and/or treating and/or restoring a normal sexual response, such as an erectile response, in SDs, such as MED, by inducing the relaxation of corpus cavernosal smooth muscle tone. Hence, the present invention provides a means to restore, or mimic, the normal erectile response.

(ii) the relaxation of corpus cavernosal smooth muscle tone through the modulation of IK_{Ca} channel activity appears to be specific to the corpus cavernosum and to have no effect on aortic smooth muscle or blood pressure in vivo. The absence of an effect in the aortic smooth muscle may be attributed to the absence of IK_{Ca} channels in cardiovascular tissue. This selective expression of IK_{Ca} channels is advantageous as it means that IK_{Ca} channel activators (such as IK_{Ca} channel openers) may be developed which can be specifically targeted to the smooth muscle in the corpus cavernosum. This selective targeting eliminates risks and side effects (such as decreases in blood pressure) which are associated with some of the vasoactive drugs which are currently used to treat erectile dysfunction.

(iii) the IK_{Ca} channel may be used as a target in high throughput screens (HTS) to identify agents capable of modulating IK_{Ca} channel activity and mediating a relaxation of corpus cavernosal smooth muscle through, for example, inhibiting calcium channel activity and/or potentiating NO-induced relaxations of corpus cavernosal smooth muscle tone.

Other advantages are discussed and are made apparent in the following commentary.

Calcium Activated Potassium Channels

As used herein the term “calcium-activated potassium channels” includes large conductance calcium activated (BK_{Ca}) channels (also referred to as Maxi K+ channels), small conductance calcium activated (SK_{Ca}) channels and intermediate conductance calcium activated (IK_{Ca}) channels which are sometimes referred to as an hSK₄ channels or IK channels or hIK₁ channels.

Currently there are three subtypes of calcium-activated potassium channels. These are large conductance calcium activated (BK_{Ca}) channels, intermediate conductance calcium activated (IK_{Ca}) channels and small conductance calcium activated (SK_{Ca}) channels. These channels are characterised by the degree of ionic conductance that passes through the channel pore during a single opening (Fan et al 1995). By way of disctinction: large conductance (BK) channels are gated by the concerted actions of internal calcium ions and membrane potential and have a unit conductance of 100 to 220 picoSiemens (pS); whereas Intermediate conductance (IK) and small conductance (SK) channels are gated solely by internal calcium ions. By way of further distinction, the IK_{Ca} and SK_{Ca} channels have a unit conductance of 20 to 85 pS and 2 to 20 pS, respectively, and are more sensitive to calcium than are BK channels. Each type of channel shows a distinct pharmacology (Ishii et al 1997).

All three of the calcium-activated potassium channels (BK_{Ca}, IK_{Ca} and SK_{Ca}) are activated by an increase in intracellular Ca²⁺. In addition, in contrast to the IK_{Ca} channels, SK_{Ca} and BK_{Ca} are additionally activated by changes in membrane potential. This is a further distinction. That is, the activation of SK_{Ca} and BK_{Ca} channels is also voltage dependent. The activation of calcium-activated potassium channels—which results in the opening of the calcium-activated potassium channels—leads to a net K⁺ efflux from the muscle cell. This loss of positive charges renders the cell interior more negative and causes a hyperpolarisation of the cell membrane which results in a decrease in membrane potential. This decrease in membrane potential inhibits voltage-activated calcium channels (VOCCs) which are activated by membrane depolarisation (Quast et al 1993) and not hyperpolarisation. Hence, the overall effect of calcium-activated potassium channel opening is a reduction in Ca²⁺ influx via VOCCs (the source of Ca²⁺ for sustained contractions) and therefore a reduction in smooth muscle tone. The hyperpolarisation induced by K⁺ channel opening has also been shown to reduce agonist-induced accumulation of inositol 1,4,5-trisphosphate (IP₃) (and hence Ca²⁺ mobilisation from intracellular stores) and the Ca²⁺ sensitivity of the contractile apparatus (Christ et al 1993). However, the mechanisms behind these observations are not fully elucidated yet.

Early reports concerning these calcium-activated potassium channels describe a lack of expression of these channels throughout the cardiovascular system and/or the central nervous system (CNS) (Bo Skanning Jensen et al 1998). However, the occurrence of these channels in the corpus cavernosum is, as yet, still unclear. By way of example, BK_{Ca} (also referred to as Maxi K⁺) channels have been shown to be involved in the NO/cGMP pathway of corporal relaxation and to play a role in maintaining the resting membrane potentials of smooth muscle. In contrast, IK_{Ca} channels have not up until now been located in the corpus cavernosum and there are no literature reports with teachings relating to the expression or function of IK_{Ca} channels in the corpus cavernosum.

Recently, literature evidence has also suggested a role for “ChTX-sensitive K⁺ channels” in horse penile arteries (Simonsen et al 1998; Prieto et al 1998) and in rabbit corpus cavernosal relaxation induced by berberine (a benzodiozolo-quinolizine alkaloid; Chiou et al 1998). Although these studies speculate on a possible role for hyperpolarising potassium channels including calcium-activated potassium conductance, neither study identifies a channel subtype responsible for corpus cavernosal relaxation. That is, neither study distinguishes between the large conductance calcium activated (BK_{Ca}) channels, the intermediate conductance calcium activated (IK_{Ca}) channels or the small conductance calcium activated (SK_{Ca}) channels. Moreover, none of the above studies suggests a possible role for these channels in the treatment of SD disorders, such as MED.

We have now surprisingly found that IK_{Ca} channels are expressed in corpus cavernosum smooth muscle cells. There are no literature reports to date relating to either IK_{Ca} channel expression in the corpus cavernosum. In addition, there are no literature reports which disclose any functional evidence for IK_{Ca} channels, such as penile IK_{Ca} channels in the smooth muscle cells of the corpus cavernosum. Any literature report relating to these channels describe a lack of expression throughout the cardiovascular system and/or the central nervous system (CNS). These findings have important implications for the treatment of SD, such as MED.

Intermediate Conductance Calcium Activated (IK_{Ca}) Channel

As used herein, the term “intermediate conductance calcium activated (IK_{Ca}) channel” refers to a subtype of the calcium activated potassium channels which is characterised by the degree of ionic conductance that passes through the channel pore during a single opening (Fan et al 1995). In contrast to the large conductance (BK) channels which are gated by the concerted actions of internal calcium ions and membrane potential and have a unit conductance of 100 to 220 picoSiemens (pS), the intermediate conductance (IK) channel is gated solely by internal calcium ions, with a unit conductance of 20 to 85 pS and is more sensitive to calcium than the BK channels.

As used herein, the term “intermediate conductance calcium-activated potassium channel” (IK_{Ca}) is used interchangeably with the term hSK4 or an active variant, homologue, derivative, fragment, part or subunit thereof. The amino acid sequence comprising hSK4 is set out as SEQ ID No 1 (Accession Number 002250) and the nucleotide sequence encoding hSK4 is set out in SEQ ID No 2 (Accession Number 002250) in the sequence listing presented herein. The term “IK_{Ca} channel” as used herein refers to the IK_{Ca} channel per se as well as amino acid sequences and/or nucleotide sequences encoding same or a variant, homologue, derivative, fragment, part or subunit thereof.

By way of background information, Joiner et al. (1997) cloned cDNAs encoding KCNN4, which they called SK4. The predicted 427-amino acid sequence of KCNN4 was approximately 40% identical to that of the rat and human SK channel proteins rSK1, rSK2, rSK3 and hSK1. Sequence analysis revealed that, like the SK channel proteins, KCNN4 contained 6 putative transmembrane domains, a conserved pore region, and a leucine zipper-like motif near the C terminus. When expressed in Chinese hamster ovary cells, KCNN4 generated a conductance of approximately 12 pS and had a very high affinity for calcium. By Northern analysis, the authors found that KCNN4 was expressed as 2.6-kb and 3.8-kb transcripts in placenta and at lower levels in lung and pancreas. On the basis of its expression pattern, physiologic properties, and low homology to other SK channel proteins, Joiner et al. (1997) proposed that KCNN4 belongs to a new subfamily of SK channels. Ishii et al. (1997) also identified a cDNA encoding KCNN4, which they called IK1.

These authors classified KCNN4 as an IK channel because its expression in Xenopus oocytes produced potassium channels with a conductance level of 39 pS that showed the biophysical and pharmacological properties of native IK channels.

Independently, Logsdon et al. (1997) isolated a human lymph node cDNA encoding KCNN4, which they called KCa4. Northern analysis revealed that KCNN4 was expressed predominantly as a 2.2-kb mRNA in a variety of tissues, with minor larger transcripts in some tissues. Logsdon et al. (1997) found that the 2.2-kb KCNN4 transcript was 10-fold more abundant in activated T cells than in resting T cells, concomitant with an increase in the KCa channel current. Expression of KCNN4 in mammalian cells produced channels having a conductance of 33 pS, with electrophysiologic properties that were very similar to those reported for the native KCa channel in activated human T lymphocytes. Logsdon et al. (1997) suggested that KCNN4 encodes the predominant KCa channel in T lymphocytes. By fluorescence in situ hybridization, Ghanshani et al. (1998) mapped the KCNN4 gene to chromosome 19q13.2.

Modulating

As used herein the term “modulating IK_{Ca} channel activity” means any one or more of: improving, increasing, enhancing, agonising, depolarising or upregulating IK_{Ca} channel activity or that the Ca²⁺ sensitivity of the IK_{Ca} channel is increased—that is, the calcium concentration required to elicit IK_{Ca} channel activity/opening is lowered. The increase in the Ca²⁺ sensitivity of the IK_{Ca} channel may be increased/enhanced by a direct or indirect opening of the IK_{Ca} channels. This increase in the Ca²⁺ sensitivity of the IK_{Ca} channel may result in a modification of the IK_{Ca} channel characteristics such that the IK_{Ca} channel opening is affected in such a way that the IK_{Ca} channel opens earlier and/or at lower intracellular calcium concentrations and/or for longer periods of time and/or with an increased open time probability.

The term “modulating IK_{Ca} channel activity” also includes the upregulation of IK_{Ca} channel expression in corpus cavernosum smooth muscle tissue such as, for example, by an agent that increases the expression of the IK_{Ca} channel and/or by the action of an agent on a substance that would otherwise impair and/or antagonise the modulation of IK_{Ca} channel activity and/or the expression of the IK_{Ca} channel.

Calcium Sensitivty

As used herein, and with reference to the IK_{Ca} channel, the term “increased Ca²⁺ sensitivity” means that the concentration of calcium ions that are required to elicit IK_{Ca} channel activation/opening is reduced/lowered. The Ca²⁺ sensitivity of the IK_{Ca} channel may be increased either (i) directly by, for example, IK_{Ca} channel activation/opening occuring at lower intracellular Ca²⁺ concentrations than one would predict from biophysical studies on the IK_{Ca} channel or (ii) indirectly by, for example, increasing the sensitivity of the IK_{Ca} channel to NO or to channel phosphorylation resulting from activation of the NO/cGMP pathway. Increasing the calcium sensitivity of the IK_{Ca} channels may lead to muscle hyperpolarisation and hence a reduction in calcium channel activity.

Inhibition of Calcium Channel Activity

As used herein, the term “inhibition of Ca²⁺ channel activity means an inhibition of channel activity which may result in either (i) a reduction in Ca²⁺ influx into smooth muscle cells and/or (ii) a reduction of Ca²⁺ mobilisation from intracellular stores and/or (ii) a decreased Ca²⁺ sensitivity of the contractile apparatus.

By way of example, an increase in intracellular Ca²⁺ may lead to an activation of Ca²⁺ activated K⁺ channels and therefore to a net efflux of K⁺ ions from the smooth muscle cells. This loss of K⁺ positive charges renders the cell interior more negative and which results in a hyperpolarisation of the cell membrane which results in a decrease in membrane potential. This decrease in membrane potential inhibits/reduces voltage-activated calcium channel (VOCC) activity which is dependent on depolarisation. The reduction in Ca²⁺ influx via VOCCs result in a reduction (and ultimate relaxation) in the source of Ca²⁺ required for sustained contractions. This results in a reduction in smooth muscle tone. The hyperpolarisation induced by K⁺ channel opening may also reduce agonist induced accumulation of inositol and hence a reduction of Ca²⁺ mobilisation from intracellular stores.

Sensitivity of IK_{Ca} Channels to NO

As used herein and with reference to the IK_{Ca} channels, the term “increased Ca²⁺ sensitivity to NO” means that NO may increase the calcium sensitivity of an IK_{Ca} channel either directly via an endogenous nitrergic pathway or indirectly via the PKG/cGMP mediated pathway.

PKG/cGMP Pathway

As used herein, the term “PKG/cGMP pathway” cyclic nucleotides, such as cGMP, are important intracellular second messengers. Nitric oxide activates soluble guanylate cyclase which results in an increase in intracellular cGMP. Intracellular cGMP is degraded by cyclic nucleotide phosphodiesterases—otherwise known as PDEs—to promote muscle relaxation. The PDEs are a family of enzymes that catalyse the degradation of cyclic nucleotides and, in doing so, are one of the cellular components that regulate the concentration of cyclic nucleotides. In recent years, at least seven PDE enzymes (such as PDEI-PDEVII), as well as many subtypes of these enzymes, have been defined based on substrate affinity and cofactor requirements (Beavo J A and Reifsnyder D H, Trends Pharmacol. Sci. 11:150 [1990]; Beavo J, In: Cyclic Nucleotide Phosphodiesterases: Structure, Regulation and Drug Action., Beavo J and Housley M D (Eds.). Wiley:Chichester, pp. 3-15 [1990]). Teachings on cyclic nucleotide phosphodiesterases can also be found in U.S. Pat. No. 5,932,423 and U.S. Pat. No. 5,932,465.

Mediating

In a preferred aspect, the agent of the present invention is capable of mediating a relaxation of corpus cavernosal smooth muscle tone.

As used herein, with reference to the relaxation of corpus cavernosal smooth muscle tone, the term “mediating” refers to a modulation of an IK_{Ca} channel activity by an agent which directly or indirectly facilitates the relaxation of corpus cavernosal smooth muscle tone. The term “mediating” also includes the potentiation of NO-induced relaxation of corpus cavernosal smooth muscle by an agent capable of modulating IK_{Ca} channel activity.

Potentiating

As used herein, with reference to the relaxation of corpus cavernosal smooth muscle tone the term “potentiating” includes any one of more of: increasing the effectiveness of an entity (such as NO), increasing the levels of an entity (such as NO), increasing the activity of an entity (such as NO), increasing the availability of an entity (such as NO), increasing the sensitivity of one entity (such as an IK_{Ca} channel) to another entity (such as NO). The potentiating effect may be a direct or an indirect effect. By way of example, the sensitivity of IK_{Ca} channels to nitric oxide (NO) may be potentiated either through increasing calcium sensitivity directly or indirectly via the PKG/cGMP pathway.

Corpus Cavernosum

As used herein, the term “corpus cavernosum” refers inter alia to a mass of tissue found in the penis. In this regard, the body of the penis is composed of three cylindrical masses of tissue, each surrounded by fibrous tissue called the tunica albuginea. The paired dorsolateral masses are called the corpora cavernosa penis (corpora=main bodies; cavernosa=hollow); the smaller midventral mass, the corpus spongiosum penis contains the spongy urethra and functions in keeping the spongy urethra open during ejaculation. All three masses are enclosed by fascia and skin and consist of erectile tissue permeated by blood sinuses. The corpus cavernosum comprises smooth muscle cells. The term “corpus cavernosum” as used herein also includes the equivalent smooth muscle cells and/or tissue in the clitoris.

Erectile Dysfunction (ED)

As used herein, the term “erectile dysfunction (ED)” includes both penile erectile dysfunction—characterised by the consistent inability of an adult male to ejaculate or to attain or hold an erection long enough for sexual intercourse—and clitoral dysfunction in the female in so far as there is substantial equivalence between penile and clitoral erectile tissue.

Penile Erection

As used herein, the term “penile erection” refers to the situation whereby, upon stimulation, which may be visual, tactile, auditory, olfactory or from the imagination, the arteries supplying the penis dilate and large quantities of blood enter the blood sinuses. Expansion of these spaces compresses the veins draining the penis, so blood outflow is slowed. These vascular changes, due to a parasympathetic reflex, result in an erection. The penis returns to its flaccid state when the arteries constrict and pressure on the veins is relieved. As used herein, the term “penile” and “penile erection” may be interpreted to apply equally to clitoris in so far as there is substantial equivalence between penile and clitoral erectile tissue.

Clitoris

As used herein, the term “clitoris” refers to the female mass of erectile tissue which is homologous to the penis in the male. Like the male structure, the clitoris is capable of enlargement upon tactile stimulation and plays a role in sexual excitement in the female. In certain types of female sexual dysfunction (FSD), such as female sexual arousal dysfunction (FSAD), the arousal dysfunction may be related to a insufficiency in genital blood flow and relaxation of clitoral corpus cavernosum.

Sexual Genitalia

As used herein, the term “sexual genitalia” refers to male and female genitalia such as the penis and clitoris.

Smooth Muscle

As used herein, the term “smooth muscle” refers to a tissue specialised for contraction composed of smooth muscle fibres (cells) which are located in the walls of hollow internal organs and innervated by autonomic motor neurons. The term “smooth muscle” means muscle lacking striations, hence giving it a smooth appearance. It is also called involuntary muscle. An increase in the concentration of Ca²⁺ in smooth muscle cytosol initiates contraction, just as in striated muscle. However, sacroplasmic reticulum (the reservoir for Ca²⁺ in striated muscle) is scanty in smooth muscle. Calcium ions flow into smooth muscle cytosol from both the extracellular fluid and sarcoplasmic reticulum, but because there are no tranverse tubules in smooth muscle fibres, it takes longer for Ca²⁺ to reach the filaments in the centre of the fibre and trigger the contractile process. This accounts, in part, for the slow onset and prolonged contraction of smooth muscle.

Contraction and Relaxation

Several mechanisms regulate contraction and relaxation of smooth muscle cells. In one, a regulatory protein called calmodulin binds to Ca²⁺ in the cytosol. Not only do calcium ions enter smooth muscle fibres slowly, but they also move slowly out of the muscle fibre when excitation declines, which delays relaxation. The prolonged presence of Ca²⁺ in the cytosol provides for smooth muscle tone, a state of continued partial contraction. Smooth muscle tissue is located in the walls of hollow internal organs such as blood vessels, airways to the lungs, the stomach, intestinal gall bladder, urinary bladder, the corpus cavernosa of the penis and the clitoris.

Targets

In one aspect of the present invention, an IK_{Ca} channel may be used as a target in screens to identify agents capable of modulating IK_{Ca} channel activity. By way of example, an IK_{Ca} channel may be used as a target in screens to identify agents capable of modulating IK_{Ca} channel activity such as, for example, increasing the Ca²⁺ sensitivity of the IK_{Ca} channel and/or the IK_{Ca} channel open time probability. In this regard, the target may comprise the amino acid sequence as set out in SEQ ID No 1 or a nucleotide sequence encoding same or a variant, homolgue, derivative or fragment thereof which is prepared by recombinant and/or synthetic means or an expression entity comprising same.

Preferably the agent increases the Ca²⁺ sensitivity of the IK_{Ca} channel and/or increases IK_{Ca} channel opening and/or increases the duration of IK_{Ca} channel opening and/or increases the probability of IK_{Ca} channel opening and/or increasing the open time probability of the IK_{Ca} channel in the range of from about 30 nM to 200 nM.

Alternatively, an IK_{Ca} channel may be used to as a target identify agents capable of mediating a relaxation of corpus cavernosal smooth muscle through the modulation of IK_{Ca} channel activity. In this respect, the target may be suitable tissue extract comprising corpus cavernosal smooth muscle cells or an equivalent therof.

The target may even be a combination of such tissue and/or recombinant targets.

Screens

Test agents capable of modulating the channel activity of IK_{Ca} targets may be screened in assays which are well known in the art. Screening may be carried out, for example in vitro, in cell culture, and/or in vivo. Biological screening assays may be based on but not limited to IK_{Ca} channel activity-based response models, binding assays (which measure how well an agent modulates IK_{Ca} channel activity), and bacterial, yeast and animal cell lines (which measure the biological effect of an agent in a smooth muscle cell, such as a corpus cavernosal smooth muscle cell or a tissue extract comprising same). The assays can be automated for high capacity-high throughput screening (HTS) in which large numbers of compounds can be tested to identify compounds with the desired IK_{Ca} channel modulating activity (see, for example WO 84/03564). Once an agent capable of modulating the IK_{Ca} channel activity—such as by modulating the opening time probability of the IK_{Ca} channel—has been identified, further steps may be carried out either to select and/or to modify compounds and/or to modify existing compounds, to improve the IK_{Ca} channel activity modulation capability.

In one preferred aspect, test agents capable of modulating the channel activity of IK_{Ca} targets are screened using the assay(s) which is (are) described by Bo Skanning Jensen et al (1998) (Characterisation of the cloned human imtermediate-conductance Ca²⁺-activated K⁺ channel Am J. Physiol 275 C848-C856).

Agent

As used herein, the term “agent” includes any entity capable of modulating an IK_{Ca} channel activity. By way of example, the agent of the present invention can include but is not limited to an IK_{Ca} channel opener, or an IK_{Ca} channel activator, agonist, enhancer or upregulator which increases the IK_{Ca} channel activity. The agent may also be an antagonist acts directly or indirectly on another entity (or target) which is capable of inhibiting/impairing an IK_{Ca} channel activity.

As used herein, the term “agent” includes, but is not limited to, a compound which may be obtainable from or produced by any suitable source, whether natural or not. The agent may be designed or obtained from a library of compounds which may comprise peptides, as well as other compounds, such as small organic molecules and particularly new lead compounds. By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a derivatised agent, a peptide cleaved from a whole protein, or a peptides synthesised synthetically (such as, by way of example, either using a peptide synthesizer or by recombinant techniques or combinations thereof, a recombinant agent, an antibody, a natural or a non-natural agent, a fusion protein or equivalent thereof and mutants, derivatives or combinations thereof. The agent may even be an IK_{Ca} channel or an amino acid sequence comprising same or a nucleotide sequence encoding same or a variant, homologue or derivative thereof or a functional equivalent thereof (such as a mimetic) or a combination of agents as outlined above.

The agent of the present invention may also be capable of displaying one or more other beneficial functional properties.

Preferably the agent may selectively agonise, and/or selectively upregulate or selectively inhibit a suitable target.

For some applications, preferably the agent has an EC₅₀ value of less than 300 nM, 250 nM, 200 nM, 150 nM, preferably less than about 100 nM, preferably less than about 75 nM, preferably less than about 50 nM, preferably less than about 25 nM, preferably less than about 20 nM, preferably less than about 15 nM, preferably less than about 10 nM, preferably less than about 5 nM.

For some applications, preferably the agent has at least about a 25, 50, 75, 100 fold selectivity to the desired target, preferably at least about a 150 fold selectivity to the desired target, preferably at least about a 200 fold selectivity to the desired target, preferably at least about a 250 fold selectivity to the desired target, preferably at least about a 300 fold selectivity to the desired target, preferably at least about a 350 fold selectivity to the desired target.

As used herein, the term “agent” may be a single entity or it may be a combination of agents.

The agent can be an amino acid sequence or a chemical derivative thereof. The substance may even be an organic compound or other chemical. The agent may even be a nucleotide sequence—which may be a sense sequence or an anti-sense sequence. The agent may even be an antibody.

If the agent is an organic compound then for some applications that organic compound will typically comprise one or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen.

The agent may contain halo groups. Here, “halo” means fluoro, chloro, bromo or iodo.

The agent may contain one or more of alkyl, alkoxy, alkenyl, alkylene and alkenylene groups—which may be unbranched- or branched-chain.

In one preferred embodiment the agent has the structure of formula (I):

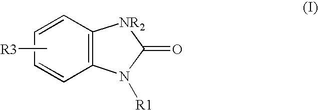

wherein

R1 is a H or a suitable substituent, such as an alkyl group which may be optionally substituted;

R2 is a H or a suitable substituent, preferably H

R3 represents one or more suitable optional substituents.

In another preferred embodiment the agent has the structure of formula (1):

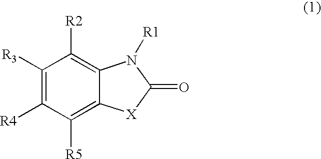

wherein:

X is selected from NR, O or S

wherein R is H or alkyl (preferably lower alkyl, more preferably C1-6 alkyl)

R1 is alkyl (preferably lower alkyl, more preferably C1-6 alkyl)

R2 is selected from H, halide, alkyl (preferably lower alkyl, more preferably C1-6 alkyl), alkoxy (preferably lower alkoxy, more preferably C1-6 alkoxy)

R3 is selected from H, halide, alkyl (preferably lower alkyl, more preferably C1-6 alkyl), alkoxy (preferably lower alkoxy, more preferably C1-6 alkoxy)

R4 is selected from H, halide, alkyl (preferably lower alkyl, more preferably C1-6 alkyl), alkoxy (preferably lower alkoxy, more preferably C1-6 alkoxy)

R5 is selected from H, halide, alkyl (preferably lower alkyl, more preferably C1-6 alkyl), alkoxy (preferably lower alkoxy, more preferably C1-6 alkoxy).

Compounds of formula (1)—wherein X═O (formula (1a)) or wherein X═S (formula (1b))—can be prepared by N-alkylation under basic conditions of the respective corresponding parent heterocycles (2a) or (2b), these in turn may be prepared by the treatment of the respective corresponding aminophenol (3a) or aminothiophenol (3b) with phosgene or another suitable carbonylating agent. Aminophenols and aminothiophenols are usually prepared from the respective corresponding nitrophenols (4a) or nitrothiophenols (4b) by reduction. Many substituted nitrophenols (4a) and nitrothiophenols (4b) are commercially available.

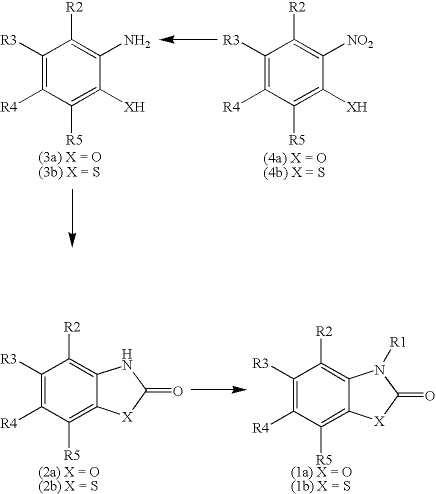

Compounds of formula 1 where X═NH (formula (1c)) can be prepared by a modification to the above scheme. In this respect, alkylation of a respective corresponding nitroaniline (5c) is carried out prior to reduction of the nitro group, providing a phenyldiamine (3c, X═NH) that is cyclised to 1c by carbonylation as described above.

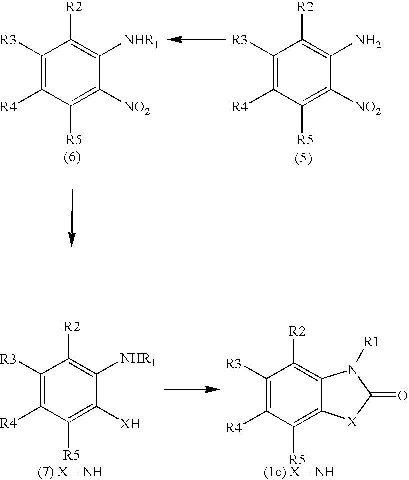

In one preferred embodiment the agent is EBIO (1-ethyl-2-benzimidazolinone) or a mimetic thereof or a pharmaceutically acceptable salt of any thereof. The structure of EBIO is:

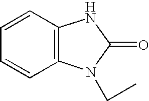

Pharmaceutically Acceptable Salt

The agent may be in the form of—and/or may be administered as—a pharmaceutically acceptable salt—such as an acid addition salt or a base salt—or a solvate thereof, including a hydrate thereof. For a review on suitable salts see Berge et al, J. Pharm. Sci., 1977, 66, 1-19.

Typically, a pharmaceutically acceptable salt may be readily prepared by using a desired acid or base, as appropriate. The salt may precipitate from solution and be collected by filtration or may be recovered by evaporation of the solvent.

Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts.

Suitable base salts are formed from bases which form non-toxic salts and examples are the sodium, potassium, aluminium, calcium, magnesium, zinc and diethanolamine salts.

Polymorphic Form(s)/Asymmetric Carbon(s)

The agent of the present invention may exist in polymorphic form.

The agent of the present invention may contain one or more asymmetric carbon atoms and therefore exists in two or more stereoisomeric forms. Where an agent contains an alkenyl or alkenylene group, cis (E) and trans (Z) isomerism may also occur. The present invention includes the individual stereoisomers of the agent and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent or a suitable salt or derivative thereof. An individual enantiomer of a compound of the agent may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

Isotopic Variations

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

Pro-drug

It will be appreciated by those skilled in the art that the agent of the present invention may be derived from a prodrug. Examples of prodrugs include entities that have certain protected group(s) and which may not possess pharmacological activity as such, but may, in certain instances, be administered (such as orally or parenterally) and thereafter metabolised in the body to form the agent of the present invention which are pharmacologically active.

Pro-moiety

It will be further appreciated that certain moieties known as “pro-moieties”, for example as described in “Design of Prodrugs” by H. Bundgaard, Elsevier, 1985 (the disclosure of which is hereby incorporated by reference), may be placed on appropriate functionalities of the agents. Such prodrugs are also included within the scope of the invention.

Agonist

In one embodiment of the present invention, preferably the agent is selected from the group consisting of an agonist, a partial agonist and a competitive agonist of an IK_{Ca} channel.

As used herein, the term “agonist” means any agent which is capable of increasing the probability of an opening of a proportion of the IK_{Ca} that is in an active form, resulting in an increased biological response. The term includes partial agonists and inverse agonists.

Antagonist

As used herein, the term “antagonist” means any agent that reduces the action of another agent, such as an agonist. The antagonist may act at on the same target as the agonist. The antagonistic action may result from a combination of the substance being antagonised (chemical antagonism) or the production of an opposite effect through a different target (functional antagonism or physiological antagonism) or as a consequence of competition for the binding site of an intermediate that links target activation to the effect observed (indirect antagonism).

Antibodies

In one embodiment of the present invention, the agent of the present invention may be an antibody. In addition, or in the alternative, the target of the present invention may be an antibody.

Antibodies may be produced by standard techniques, such as by immunisation with the substance of the invention or by using a phage display library.

For the purposes of this invention, the term “antibody”, unless specified to the contrary, includes but is not limited to, polyclonal, monoclonal, chimeric, single chain, Fab fragments and fragments produced by a Fab expression library. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. Furthermore, the antibodies and fragments thereof may be humanised antibodies. Neutralizing antibodies, i.e., those which inhibit biological activity of the substance polypeptides, are especially preferred for diagnostics and therapeutics.

If polyclonal antibodies are desired, a selected mammal (e.g., mouse, rabbit, goat, horse, etc.) is immunised with an immunogenic polypeptide bearing a epitope(s) obtainable from an identified agent and/or substance of the present invention. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance polypeptide is administered to immunologically compromised individuals for the purpose of stimulating systemic defence.

Serum from the immunised animal is collected and treated according to known procedures. If serum containing polyclonal antibodies to an epitope obtainable from an identifed agent and/or substance of the present invention contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against epitopes obtainable from an identifed agent and/or substance of the present invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; i.e., for isotype and epitope affinity.

Monoclonal antibodies to the substance and/or identified agent of the present invention may be prepared using any technique which provides for the production of antibody molecules by continuous cell lines in culture. These include, but are not limited to, the hybridoma technique originally described by Koehler and Milstein (1975 Nature 256:495-497), the human B-cell hybridoma technique (Kosbor et al (1983) Immunol Today 4:72; Cote et al (1983) Proc Natl Acad Sci 80:2026-2030) and the EBV-hybridoma technique (Cole et al (1985) Monoclonal Antibodies and Cancer Therapy, Alan R Liss Inc, pp 77-96). In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,779) can be adapted to produce the substance specific single chain antibodies.

Antibodies, both monoclonal and polyclonal, which are directed against epitopes obtainable from an identifed agent and/or substance of the present invention are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the substance and/or agent against which protection is desired. Techniques for raising anti-idiotype antibodies are known in the art. These anti-idiotype antibodies may also be useful in therapy.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci 86: 3833-3837), and Winter G and Milstein C (1991; Nature 349:293-299).

Antibody fragments which contain specific binding sites for the substance may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse W D et al (1989) Science 256:1275-128 1).

Chemical Synthesis Methods

Typically the agent of the present invention will be prepared by chemical synthesis techniques.

The agent of the present invention or variants, homologues, derivatives, fragments or mimetics thereof may be produced using chemical methods to synthesize the agent in whole or in part. For example, peptides can be synthesized by solid phase techniques, cleaved from the resin, and purified by preparative high performance liquid chromatography (e.g., Creighton (1983) Proteins Structures And Molecular Principles, WH Freeman and Co, New York N.Y.). The composition of the synthetic peptides may be confirmed by amino acid analysis or sequencing (e.g., the Edman degradation procedure; Creighton, supra).

Direct synthesis of the agent or variants, homologues, derivatives, fragments or mimetics thereof can be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences comprising the agent or any part thereof, may be altered during direct synthesis and/or combined using chemical methods with a sequence from other subunits, or any part thereof, to produce a variant agent, such as, for example, a variant IK_{Ca} channel.

In an alternative embodiment of the invention, the coding sequence of the agent or variants, homologues, derivatives, fragments or mimetics thereof may be synthesized, in whole or in part, using chemical methods well known in the art (see Caruthers M H et al (1980) Nuc Acids Res Symp Ser 215-23, Horn T et al (1980) Nuc Acids Res Symp Ser 225-232).

Mimetic

As used herein, the term “mimetic” relates to any chemical which includes, but is not limited to, a peptide, polypeptide, antibody or other organic chemical which has the same qualitative activity or effect as a known agent. That is, a mimetic may be a functional equivalent of a known agent, (such as 1-ethyl-2-benzimidazolinone (EBIO)) which is capable of increasing the open time probability of an IK_{Ca} channel or it may be a functional equivalent of an IK_{Ca} channel found in corpus cavernosal smooth muscle tissue.

Derivative

The term “derivative” or “derivatised” as used herein includes chemical modification of an agent. Illustrative of such chemical modifications would be replacement of hydrogen by a halo group, an alkyl group, an acyl group or an amino group.

Chemical Modification

In one embodiment of the present invention, the agent may be a chemically modified agent.

The chemical modification of an agent of the present invention may either enhance or reduce hydrogen bonding interaction, charge interaction, hydrophobic interaction, Van Der Waals interaction or dipole interaction between the agent and the target.

In one aspect, the identified agent may act as a model (for example, a template) for the development of other compounds.

Recombinant Methods

Typically the agent of the present invention is prepared by recombinant DNA techniques.

In one embodiment, preferably the agent is an IK_{Ca} channel.

Preferably the IK_{Ca} channel is prepared by recombinant DNA techniques.

Preferably the IK_{Ca} channel of the present invention comprises the amino acid sequence set out in SEQ ID No 1 or a variant, homologue, derivative or fragment thereof.

Amino Acid Sequences

As used herein, the term “amino acid sequence” refers to peptide, polypeptide sequences, protein sequences or portions thereof.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

The amino acid sequence may be prepared isolated from a suitable source, or it may be made synthetically or it may be prepared by use of recombinant DNA techniques.

In one aspect, the present invention provides an amino acid sequence that is capable of acting as a target in an assay for the identification of one or more agents and/or derivatives thereof capable of affecting the amino acid sequence in order to modulate IK_{Ca} channel activity.

Preferably the target is an IK_{Ca} channel.

Preferably, the IK_{Ca} channel is an isolated IK_{Ca} channel and/or purified and/or non-native IK_{Ca} channel.

The IK_{Ca} channel of the present invention may be in a substantially isolated form. It will be understood that the IK_{Ca} channel may be mixed with carriers or diluents which will not interfere with the intended purpose of the IK_{Ca} channel and still be regarded as substantially isolated. The IK_{Ca} channel of the present invention may also be in a substantially purified form, in which case it will generally comprise the IK_{Ca} channel in a preparation in which more than 90%, e.g. 95%, 98% or 99% of the IK_{Ca} channel in the preparation is a peptide comprising SEQ ID No 1 or variants, homologues, derivatives or fragments thereof.

Variants/Homologues/Derivatives of Amino Acid Sequences

Preferred amino acid sequences of the present invention are set out in SEQ ID No 1 are sequences obtainable from the IK_{Ca} channel of the present invention but also include homologous sequences obtained from any source and for example, synthetic peptides, as well as variants or derivatives thereof.

Thus, the present invention covers variants, homologues or derivatives of the amino acid sequences presented herein, as well as variants, homologues or derivatives of the nucleotide sequence coding for those amino acid sequences.

In the context of the present invention, a homologous sequence is taken to include an amino acid sequence which is at least 75, 85 or 90% identical, preferably at least 95 or 98% identical at the amino acid level over at least, for example, the amino acid sequence as set out in SEQ ID No 1 of the sequence listing herein. In particular, homology should typically be considered with respect to those regions of the sequence known to be essential for IK_{Ca} channel activity rather than non-essential neighbouring sequences. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al 1984, Nucleic Acids Research 12:387). Examples of other software than can perform sequence comparisons include, but are not limited to, the BLAST package (see Ausubel et al 1999 ibid—Chapter 18), FASTA (Atschul et al 1990, J. Mol. Biol., 403-410) and the GENEWORKS suite of comparison tools. Both BLAST and FASTA are available for offline and online searching (see Ausubel et al 1999 ibid, pages 7-58 to 7-60). However it is preferred to use the GCG Bestfit program. A new tool, called BLAST 2 Sequences is also available for comparing protein and nucleotide sequence (see FEMS Microbiol Left 1999 174(2): 247-50; FEMS Microbiol Lett 1999 177(1): 187-8 and tatiana@ncbi.nlm.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The terms “variant” or “derivative” in relation to the amino acid sequences of the present invention includes any substitution of, variation of, modification of, replacement of, deletion of or addition of one (or more) amino acids from or to the sequence providing the resultant amino acid sequence has IK_{Ca} channel activity, preferably having at least the same IK_{Ca} channel activity as the amino acid sequence set out in SEQ ID No 1 of the sequence listing presented herein.

SEQ ID No 1 of the sequence listing herein may be modified for use in the present invention. Typically, modifications are made that maintain the binding specificity of the sequence. Amino acid substitutions may be made, for example from 1, 2 or 3 to 10 or 20 substitutions provided that the modified sequence retains the required IK_{Ca} channel activity. Amino acid substitutions may include the use of non-naturally occurring analogues.

The IK_{Ca} channel of the present invention may also have deletions, insertions or substitutions of amino acid residues which produce a silent change and result in a functionally equivalent IK_{Ca} channel. Deliberate amino acid substitutions may be made on the basis of similarity in polarity, charge, solubility, hydrophobicity, hydrophilicity, and/or the amphipathic nature of the residues as long as the activity of the IK_{Ca} channel is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Preferably, the isolated IK_{Ca} channel and/or purified IK_{Ca} channel and/or non-native IK_{Ca} channel is prepared by use of recombinant techniques.

Nucleotide Sequence

As used herein, the term “nucleotide sequence” is synonymous with the term “polynucleotide”.

The nucleotide sequence may be DNA or RNA of genomic or synthetic or of recombinant origin. The nucleotide sequence may be double-stranded or single-stranded whether representing the sense or antisense strand or combinations thereof.

For some applications, preferably, the nucleotide sequence is DNA.

For some applications, preferably, the nucleotide sequence is prepared by use of recombinant DNA techniques (e.g. recombinant DNA).

For some applications, preferably, the nucleotide sequence is cDNA.

For some applications, preferably, the nucleotide sequence may be the same as the naturally occurring form.

In one aspect, the present invention provides a nucleotide sequence encoding a substance capable of acting as a target in an assay (such as a yeast two hybrid assay) for the identification of one or more agents and/or derivatives thereof capable of modulating IK_{Ca} channel activity.

In one aspect of the present invention, the nucleotide sequence encodes an IK_{Ca} channel.

It will be understood by a skilled person that numerous different nucleotide sequences can encode the same IK_{Ca} channel of the present invention as a result of the degeneracy of the genetic code. In addition, it is to be understood that skilled persons may, using routine techniques, make nucleotide substitutions that do not affect the IK_{Ca} channel encoded by the nucleotide sequence of the present invention to reflect the codon usage of any particular host organism in which the IK_{Ca} channel of the present invention is to be expressed. The terms “variant”, “homologue” or “derivative” in relation to the nucleotide sequence set out in SEQ ID No 2 of the present invention include any substitution of, variation of, modification of, replacement of, deletion of or addition of one (or more) nucleic acid from or to the sequence providing the resultant nucleotide sequence encoding the IK_{Ca} channel has an IK_{Ca} channel activity, preferably having at least the same IK_{Ca} channel activity as that encoded by the nucleotide sequence set out in SEQ ID No 2 the sequence listings of the present invention.

As indicated above, with respect to sequence homology, preferably there is at least 75%, more preferably at least 85%, more preferably at least 90% homology to the sequences shown in the sequence listing herein. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

The present invention also encompasses nucleotide sequences that are capable of hybridising selectively to the sequences presented herein, or any variant, fragment or derivative thereof, or to the complement of any of the above. Nucleotide sequences are preferably at least 15 nucleotides in length, more preferably at least 20, 30, 40 or 50 nucleotides in length.

Hybridisation

The term “hybridization” as used herein shall include “the process by which a strand of nucleic acid joins with a complementary strand through base pairing” as well as the process of amplification as carried out in polymerase chain reaction (PCR) technologies.

Nucleotide sequences of the invention capable of selectively hybridising to the nucleotide sequences presented herein, or to their complement, will be generally at least 75%, preferably at least 85 or 90% and more preferably at least 95% or 98% homologous to the corresponding complementary nucleotide sequence presented herein over a region of at least 20, preferably at least 25 or 30, for instance at least 40, 60 or 100 or more contiguous nucleotides. Preferred nucleotide sequences of the invention will comprise regions homologous to the nucleotide sequence set out in SEQ ID No 2 of the sequence listings of the present invention preferably at least 80 or 90% and more preferably at least 95% homologous to the nucleotide sequence set out in SEQ ID No 2 of the sequence listings of the present invention.

The term “selectively hybridizable” means that the nucleotide sequence, when used as a probe, is used under conditions where a target nucleotide sequence of the invention is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC {1×SSC=0.15 M NaCl, 0.015 M Na₃ Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Nucleotide sequences which are not 100% homologous to the sequences of the present invention but fall within the scope of the invention can be obtained in a number of ways. Other variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the nucleotide sequence set out in SEQ ID No 2 of the sequence listings of the present invention under conditions of medium to high stringency. Similar considerations apply to obtaining species homologues and allelic variants of the amino acid and/or nucleotide sequences of the present invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences, such as the nucleotide sequence set out in SEQ ID No 2 of the sequence listings of the present invention. This may be useful where for example silent codon changes are required to sequences to optimise codon preferences for a particular host cell in which the nucleotide sequences are being expressed. Other sequence changes may be desired in order to introduce restriction enzyme recognition sites, or to alter the IK_{Ca} channel activity of the IK_{Ca} channel encoded by the nucleotide sequences.

The nucleotide sequences of the present invention may be used to produce a primer, e.g. a PCR primer, a primer for an alternative amplification reaction, a probe e.g. labelled with a revealing label by conventional means using radioactive or non-radioactive labels, or the nucleotide sequences may be cloned into vectors. Such primers, probes and other fragments will be at least 15, preferably at least 20, for example at least 25, 30 or 40 nucleotides in length, and are also encompassed by the term nucleotide sequence of the invention as used herein.

The nucleotide sequences such as a DNA polynucleotides and probes according to the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Due to the inherent degeneracy of the genetic code, other DNA sequences which encode substantially the same or a functionally equivalent amino acid sequence, may be used to clone and express the IK_{Ca} channel. As will be understood by those of skill in the art, for certain expression systems, it may be advantageous to produce the IK_{Ca} channel—encoding nucleotide sequences possessing non-naturally occurring codons. Codons preferred by a particular prokaryotic or eukaryotic host (Murray E et al (1989) Nuc Acids Res 17:477-508) can be selected, for example, to increase the rate of the IK_{Ca} channel expression or to produce recombinant RNA transcripts having desirable properties, such as a longer half-life, than transcripts produced from naturally occurring sequence.

Vector

In one embodiment of the present invention, an agent of the present invention or an IK_{Ca} channel may be administered directly to an individual.

In another embodiment of the present invention, a vector comprising a nucleotide sequence encoding an agent of the present invention or an IK_{Ca} channel is administered to an individual.

Preferably the recombinant IK_{Ca} channel is prepared and/or delivered to a target site using a genetic vector.

As it is well known in the art, a vector is a tool that allows or faciliates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a host and/or a target cell for the purpose of replicating the vectors comprising the nucleotide sequences of the present invention and/or expressing the proteins of the invention encoded by the nucleotide sequences of the present invention. Examples of vectors used in recombinant DNA techniques include but are not limited to plasmids, chromosomes, artificial chromosomes or viruses.

The term “vector” includes expression vectors and/or transformation vectors.

The term “expression vector” means a construct capable of in vivo or in vitro/ex vivo expression.

The term “transformation vector” means a construct capable of being transferred from one species to another.

“Naked DNA”

The vectors comprising nucleotide sequences encoding an agent of the present invention or an IK_{Ca} channel of the present invention for use in treating SDs such as MED may be administered directly as “a naked nucleic acid construct”, preferably further comprising flanking sequences homologous to the host cell genome.

As used herein, the term “naked DNA” refers to a plasmid comprising a nucleotide sequences encoding an agent of the present invention or a IK_{Ca} channel of the present invention together with a short promoter region to control its production. It is called “naked” DNA because the plasmids are not carried in any delivery vehicle. When such a DNA plasmid enters a host cell, such as a eukaryotic cell, the proteins it encodes (such as an agent of the present invention or an IK_{Ca} channel) are transcribed and translated within the cell.

Non-viral Delivery

Alternatively, the vectors comprising nucleotide sequences of the present invention or an agent of the present invention may be introduced into suitable host cells using a variety of non-viral techniques known in the art, such as transfection, transformation, electroporation and biolistic transformation.

As used herein, the term “transfection” refers to a process using a non-viral vector to deliver a gene to a target mammalian cell.

Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology 1996 14; 556), multivalent cations such as spermine, cationic lipids or polylysine, 1, 2,-bis (oleoyloxy)-3-(trimethylammonio) propane (DOTAP)-cholesterol complexes (Wolff and Trubetskoy 1998 Nature Biotechnology 16: 421) and combinations thereof.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Viral Vectors

Alternatively, the vectors comprising an agent of the present invention or nucleotide sequences of the present invention may be introduced into suitable host cells using a variety of viral techniques which are known in the art, such as for example infection with recombinant viral vectors such as retroviruses, herpes simplex viruses and adenoviruses.

Preferably the vector is a recombinant viral vectors. Suitable recombinant viral vectors include but are not limited to adenovirus vectors, adeno-associated viral (AAV) vectors, herpes-virus vectors, a retroviral vector, lentiviral vectors, baculoviral vectors, pox viral vectors or parvovirus vectors (see Kestler et al 1999 Human Gene Ther 10(10):1619-32). In the case of viral vectors, delivery of the nucleotide sequence encoding the IK_{Ca} channel is mediated by viral infection of a target cell.

Targeted Vector

The term “targeted vector” refers to a vector whose ability to infect/transfect/transduce a cell or to be expressed in a host and/or target cell is restricted to certain cell types within the host organism, usually cells having a common or similar phenotype.

Replication Vectors

The nucleotide sequences encoding an agent of the present invention or the IK_{Ca} channel of the present invention may be incorporated into a recombinant replicable vector. The vector may be used to replicate the nucleotide sequence in a compatible host cell. Thus in one embodiment of the present invention, the invention provides a method of making the IK_{Ca} channel of the present invention by introducing a nucleotide sequence of the present invention into a replicable vector, introducing the vector into a compatible host cell, and growing the host cell under conditions which bring about replication of the vector. The vector may be recovered from the host cell.

Expression Vector

Preferably, an agent of the present invention or a nucleotide sequence of present invention which is inserted into a vector is operably linked to a control sequence that is capable of providing for the expression of the coding sequence, such as the coding sequence of the IK_{Ca} channel of the present invention by the host cell, i.e. the vector is an expression vector. An agent of the present invention or an IK_{Ca} channel produced by a host recombinant cell may be secreted or may be contained intracellularly depending on the sequence and/or the vector used. As will be understood by those of skill in the art, expression vectors containing an agent of the present invention or the IK_{Ca} channel coding sequences can be designed with signal sequences which direct secretion of the agent of the present invention or the IK_{Ca} channel coding sequences through a particular prokaryotic or eukaryotic cell membrane.

Expression in vitro

The vectors of the present invention may be transformed or transfected into a suitable host cell and/or a target cell as described below to provide for expression of an agent of the present invention or an IK_{Ca} channel of the present invention.

This process may comprise culturing a host cell and/or target cell transformed with an expression vector under conditions to provide for expression by the vector of a coding sequence encoding an agent of the present invention or the IK_{Ca} channel and optionally recovering the expressed agent of the present invention or IK_{Ca} channel. The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. The expression of an agent of the present invention or an IK_{Ca} channel of the invention may be constitutive such that they are continually produced, or inducible, requiring a stimulus to initiate expression. In the case of inducible expression, production of an agent of the present invention or an IK_{Ca} channel can be initiated when required by, for example, addition of an inducer substance to the culture medium, for example dexamethasone or IPTG.

Fusion Proteins

The IK_{Ca} channel or an agent of the present invention may be expressed as a fusion protein to aid in extraction and purification and/or delivery of the agent of the present invention or the IK_{Ca} channel to an individual and/or to facilitate the development of a screen for agents capable of modulating IK_{Ca} channel activity. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the activity of the target.

The fusion protein may comprise an antigen or an antigenic determinant fused to the substance of the present invention. In this embodiment, the fusion protein may be a non-naturally occurring fusion protein comprising a substance which may act as an adjuvant in the sense of providing a generalised stimulation of the immune system. The antigen or antigenic determinant may be attached to either the amino or carboxy terminus of the substance.

In another embodiment of the invention, the amino acid sequence may be ligated to a heterologous sequence to encode a fusion protein. For example, for screening of peptide libraries for agents capable of affecting the substance activity, it may be useful to encode a chimeric substance expressing a heterologous epitope that is recognized by a commercially available antibody.

Host Cells

A wide variety of host cells can be employed for expression of the nucleotide sequences encoding the agent—such as an agent of the present invention or an IK_{Ca} channel of the present invention. These cells may be both prokaryotic and eukaryotic host cells. Suitable host cells include bacteria such as E coli, yeast, filamentous fungi, insect cells, mammalian cells, typically immortalized, e.g., mouse, CHO, human and monkey cell lines and derivatives thereof.

Examples of suitable expression hosts within the scope of the present invention are fungi such as Aspergillus species (such as those described in EP-A-0184438 and EP-A-0284603) and Trichoderma species; bacteria such as Bacillus species (such as those described in EP-A-0134048 and EP-A-0253455), Streptomyces species and Pseudomonas species; and yeasts such as Kluyveromyces species (such as those described in EP-A-0096430 and EP-A-0301670) and Saccharomyces species. By way of example, typical expression hosts may be selected from Aspergillus niger, Aspergillus niger var. tubigenis, Aspergillus niger var. awamori, Aspergillus aculeatis, Aspergillus nidulans, Aspergillus orvzae, Trichoderma reesei, Bacillus subtilis, Bacillus licheniformis, Bacillus amyloliquefaciens, Kluyveromyces lactis and Saccharomyces cerevisiae.

The use of suitable host cells—such as yeast, fungal and plant host cells—may provide for post-translational modifications (e.g. myristoylation, glycosylation, truncation, lapidation and tyrosine, serine or threonine phosphorylation) as may be needed to confer optimal biological activity on recombinant expression products of the present invention.

Preferred host cells are able to process the expression products to produce an appropriate mature polypeptide. Examples of processing includes but is not limited to glycosylation, ubiquitination, disulfide bond formation and general post-translational modification.

Pharmaceutical Compositions

The present invention also provides a pharmaceutical composition comprising a therapeutically effective amount of the agent of the present invention and a pharmaceutically acceptable carrier, diluent or excipients (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, the agents of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

In a preferred embodiment, the asents of the present invention are delivered systemically (such as orally, buccally, sublingually), more preferably orally.

Hence, preferably the agent is in a form that is suitable for oral delivery.

For some embodiments, preferably the agent—when in use—does not act on the central nervous system.

For some embodiments, preferably the agent—when in use—is peripherally acting.

Administration

The term “administered” includes delivery by viral or non-viral techniques. Viral delivery mechanisms include but are not limited to adenoviral vectors, adeno-associated viral (AAV) vectos, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The agents of the present invention may be administered alone but will generally be administered as a pharmaceutical composition—e.g. when the agent is in admixture with a suitable pharmaceutical excipient, diluent or carrier selected with regard to the intended route of administration and standard pharmaceutical practice.

For example, the agent can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of:

oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, via the pensis, vaginal, epidural, sublingual.

It is to be understood that not all of the agent need be administered by the same route. Likewise, if the composition comprises more than one active component, then those components may be administered by different routes.

If the agent of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the agent; and/or by using infusion techniques.

For parenteral administration, the agent is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the agent of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the agent of the present invention can be administered in the form of a suppository or pessary, or it may be applied topically in the form of a gel, hydrogel, lotion, solution, cream, ointment or dusting powder. The agent of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compositions of the present invention may be administered by direct injection.

For some applications, preferably the agent is administered orally.

For some applications, preferably the agent is administered topically.

Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The agent and/or the pharmaceutical composition of the present invention may be administered in accordance with a regimen of from 1 to 10 times per day, such as once or twice per day.

For oral and parenteral administration to human patients, the daily dosage level of the agent may be in single or divided doses.

Depending upon the need, the agent may be administered at a dose of from 0.01 to 30 mg/kg body weight, such as from 0.1 to 10 mg/kg, more preferably from 0.1 to 1 mg/kg body weight. Naturally, the dosages mentioned herein are exemplary of the average case. There can, of course, be individual instances where higher or lower dosage ranges are merited.

Formulation

The agents of the present invention may be formulated into a pharmaceutical composition, such as by mixing with one or more of a suitable carrier, diluent or excipient, by using techniques that are known in the art.

The following present some non-limiting examples of formulations.

Formulation 1: A tablet is prepared using the following ingredients:

| | weight/mg |
|---|---|
| Agent | 250 |
| Cellulose, microcrystalline | 400 |
| Silicon dioxide, fumed | 10 |
| Stearic acid | 5 |
| Total | 665 |

the components are blended and compressed to form tablets each weighing 665 mg.

Formulation 2: An intravenous formulation may be prepared as follows:

| | |
|---|---|
| Agent | 100 mg |
| Isotonic saline | 1,000 ml |

Individual

As used herein, the term “individual” refers to vertebrates, particularly members of the mammalian species. The term includes but is not limited to domestic animals, sports animals, primates and humans.

Treatment

It is to be appreciated that all references herein to treatment include curative, palliative and prophylactic treatment.

Pharmaceutical Combinations

In general, the agent may be used in combination with one or more other pharmaceutically active agents. The other agent is sometimes referred to as being an auxiliary agent. Examples of auxiliary agents include a potentiator of intracellular cGMP (such a phosphodiestertase type 5 inhibitor eg Sildenafil, or a nitric oxide donor, or a nitric oxide precursor eg L-arginine) and/or a centrally acting pharmaceutical (e.g. a dopamine receptor or melanocortin receptor agonist, such as apomorphine or melanotan II). Teachings on the use of apomorphine as a pharmaceutical may be found in U.S. Pat. No. 5,945,117. In that particular document, apomorphine is delivered sub-lingually. In addition, or in the alternative, the agent may be used in combination with one or more of: a PDE5 inhibitor (eg sildenafil, vardenafil (Bayer BA 38-9456) and IC351 (Cialis, Icos Lilly)), one or more of a nitric oxide donor (eg NMI-921), one or more of a dopamine receptor agonist (eg apomorphine, Uprima, Ixsene), one or more of a melanocortin receptor agonist (eg Melanotan II or PT14), one or more of a potassium channel opener (eg a K_{ATP} channel opener (eg minoxidil, nicorandil) and/or a calcium activated potassium channel opener (eg BMS-204352), one or more of a al-adrenoceptor antagonist (eg phentolamine, Vasofem, Vasomax), one or more of a VIP receptor agonist or a VIP analogue (eg Ro-125-1553) or a VIP fragment, one or more of a α-adrenoceptor antagonist with VIP combination (eg Invicorp, Aviptadil), one or more of a α2-adrenoceptor antagonist (eg yohimbine), one or more of a estrogen, estrogen and medroxyprogesterone or medroxyprogesterone acetate (MPA) or oestrogen and methyl testosterone hormone replacement therapy agent (eg HRT especially Premarin, Cenestin, Oestrofeminal, Equin, Estrace, Estrofem, Elleste Solo, Estring, Eastraderm, Eastraderm TTS, Eastraderm Matrix, Dermestril, Premphase, Prempro, Prempak, Premique, Estratest, Estratest HS, Tibolone), one or more of a testosterone replacement agent (inc DHEA (dehydroandrostendione), testosterone (Tostrelle) or a testosterone implant (Organon)), one or more of a testosterone/oestradiol agent one or more of an estrogen agonists, one or more of a serotonin receptor agonist or antagonist (eg 5HT1A, 5HT2C, 5HT2A and 5HT3 receptor agonists and antagonists; as described in WO2000/28993), one or more of a prostanoid receptor agonist (eg Muse, alprostadil, misoprostol), one or more of a purinergic receptor agonist one or more antidepressant agents (eg bupropion (Wellbutrin), mirrtazapine, nefazodone).

The structure of IC351 is:

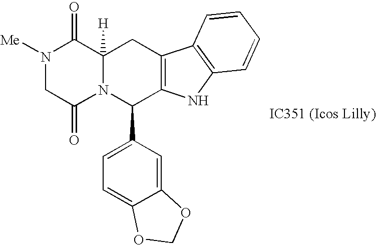

In more detail, the present invention further comprises the combination of a compound of the invention for the treatment of male sexual dysfunction as outlined herein (more particularly male erectile dysfunction) with one or more of the following auxiliary active agents. The combination provides a treatment for erectile dysfunctions of organic, neurogenic and/or psychogenic origin. The combinations may also have the ability to treat hypoactive sexual desire disorders, sexual arousal disorders, anorgasmic and sexual pain disorders.

Thus a further aspect of the invention provides a pharmaceutical combination (for simultaneous, separate or sequential administration) comprising a compound of the invention and one or more of the following auxiliary active agents:

1) one or more naturally occurring or synthetic prostaglandins or esters thereof. Suitable prostaglandins for use herein include compounds such as alprostadil, prostaglandin E₁, prostaglandin E₀, 13,14-dihydroprosta glandin E₁, prostaglandin E₂, eprostinol, natural synthetic and semi-synthetic prostaglandins and derivatives thereof including those described in WO-00033825 and/or U.S. Pat. No. 6,037,346 issued on 14th Mar. 2000 all incorporated herein by reference, PGE₀, PGE₁, PGA₁, PGB₁, PGF₁α, 19-hydroxy PGA₁, 19-hydroxy-PGB₁, PGE₂, PGB₂, 19-hydroxy-PGA₂, 19-hydroxy-PGB₂, PGE₃α, carboprost tromethamine dinoprost, tromethamine, dinoprostone, lipo prost, gemeprost, metenoprost, sulprostune, tiaprost and moxisylate;

2) one or more α-adrenergic receptor antagonist compounds also known as α-adrenoceptors or α-receptors or α-blockers. Suitable compounds for use herein include: the α-adrenergic receptor blockerss as described in PCT application WO99/30697 published on 14th Jun. 1998, the disclosures of which relating to α-adrenergic receptors are incorporated herein by reference and include, selective α₁-adrenoceptor or α₂-adrenoceptor blockers and non-selective adrenoceptor blockers, suitable α₁-adrenoceptor blockers include: phentolamine, phentolamine mesylate, trazodone, alfuzosin, indoramin, naftopidil, tamsulosin, dapiprazole, phenoxybenzamine, idazoxan, efaraxan, yohimbine, rauwolfa alkaloids, Recordati 15/2739, SNAP 1069, SNAP 5089, RS17053, SL 89.0591, doxazosin, terazosin, abanoquil and prazosin; α₂-blocker blockers from U.S. Pat. No. 6,037,346 [14th Mar. 2000] dibenarnine, tolazoline, trimazosin and dibenarnine; α-adrenergic receptors as described in U.S. Pat. Nos. 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference; α₂-Adrenoceptor blockers include: clonidine, papaverine, papaverine hydrochloride, optionally in the presence of a cariotonic agent such as pirxamine;

3) one or more NO-donor (NO-agonist) compounds. Suitable NO-donor compounds for use herein include organic nitrates, such as mono- di or tri-nitrates or organic nitrate esters including glyceryl trinitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine molsidomine, S-nitroso-N-acetyl penicilliamine (SNAP) S-nitroso-N-glutathione (SNO-GLU), N-hydroxy-L-arginine, amylnitrate, linsidomine, linsidomine chlorohydrate, (SIN-1) S-nitroso-N-cysteine, diazenium diolates,(NONOates), 1,5-pentanedinitrate, L-arginene, ginseng, zizphi fructus, molsidomine, Re-2047, nitrosylated maxisylyte derivatives such as NMI-678-11 and NMI-937 as described in published PCT application WO 0012075;

4) one or more potassium channel openers or modulators. Suitable potassium channel openers/modulators for use herein include nicorandil, cromokalim, levcromakalim, lemakalim, pinacidil, cliazoxide, minoxidil, charybdotoxin, glyburide, 4-amini pyridine, BaCl₂;

5) one or more dopaminergic agents, preferably apomorphine or a selective D2, D3 or D2/D₃agonist such as, pramipexole and ropirinol (as claimed in WO-0023056), PNU95666 (as claimed in WO-0040226);

6) one or more vasodilator agents. Suitable vasodilator agents for use herein include nimodepine, pinacidil, cyclandelate, isoxsuprine, chloroprumazine, halo peridol, Rec 15/2739, trazodone;

7) one or more thromboxane A2 agonists;

8) one or more CNS active agents;

9) one or more ergot alkoloids; Suitable ergot alkaloids are described in U.S. Pat. No. 6,037,346 issued on 14th Mar. 2000 and include acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride and terguride;

10) one or more compounds which modulate the action of natruretic factors in particular atrial naturetic factor (also known as atrial naturetic peptide), B type and C type naturetic factors such as inhibitors or neutral endopeptidase;

11) one or more compounds which inhibit angiotensin-converting enzyme such as enapril, and combined inhibitors of angiotensin-converting enzyme and neutral endopeptidase such as omapatrilat.

12) one or more angiotensin receptor antagonists such as losartan;

13) one or more substrates for NO-synthase, such as L-arginine;

14) one or more calcium channel blockers such as amlodipine;

15) one or more antagonists of endothelin receptors and inhibitors or endothelin-converting enzyme;

16) one or more cholesterol lowering agents such as statins (e.g. atorvastatin/Lipitor-trade mark) and fibrates;

17) one or more antiplatelet and antithrombotic agents, e.g. tPA, uPA, warfarin, hirudin and other thrombin inhibitors, heparin, thromboplastin activating factor inhibitors;

18) one or more insulin sensitising agents such as rezulin and hypoglycaemic agents such as glipizide;

19) L-DOPA or carbidopa;

20) one or more acetylcholinesterase inhibitors such as donezipil;

21) one or more steroidal or non-steroidal anti-inflammatory agents;

22) one or more estrogen receptor modulators and/or estrogen agonists and/or estrogen antagonists, preferably raloxifene or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof the preparation of which is detailed in WO 96/21656;

23) one or more of a PDE inhibitor, more particularly a PDE 2, 3, 4, 5, 7 or 8 inhibitor, preferably PDE2 or PDE5 inhibitor and most preferably a PDE5 inhibitor (see hereinafter), said inhibitors preferably having an IC50 against the respective enzyme of less than 100 nM;

24) one or more of an NPY (neuropeptide Y) inhibitor, more particularly NPY1 or NPY5 inhibitor, preferably NPY1 inhibitor; preferably said NPY inhibitors (including NPY1 and NPY5) have an IC50 of less than 100 nM, more preferably less than 50 nM;

25) one or more of a NEP inhibitor preferably having an IC50 for NEP of less than 300 nM, more preferably less than 100 nM;

26) one or more of vasoactive intestinal protein (VIP), VIP mimetic, VIP analogue, more particularly mediated by one or more of the VIP receptor subtypes VPAC1,VPAC or PACAP (pituitory adenylate cyclase activating peptide), one or more of a VIP receptor agonist or a VIP analogue (eg Ro-125-1553) or a VIP fragment, one or more of a α-adrenoceptor antagonist with VIP combination (eg Invicorp, Aviptadil);

27) one or more of a melanocortin receptor agonist or modulator or melanocortin ehancer, such as melanotan II, PT-14, PT-141 or compounds claimed in WO-09964002, WO-00074679, WO-09955679, WO-00105401, WO-00058361, WO-00114879, WO-00113112, WO-09954358;

28) one or more of a serotonin receptor agonist, antagonist or modulator, more particularly agonists, antagonists or modulators for 5HT1A (including VML 670), 5HT2A, 5HT2C, 5HT3 and/or 5HT6 receptors, including those described in WO-09902159, WO-00002550 and/or WO-00028993;

29) one or more of a testosterone replacement agent (inc dehydroandrostendione), testosternone (Tostrelle), dihydrotestosterone or a testosterone implant;

30) one or more of estrogen, estrogen and medroxyprogesterone or medroxyprogesterone acetate (MPA) (i.e. as a combination), or estrogen and methyl testosterone hormone replacement therapy agent (e.g. HRT especially Premarin, Cenestin, Oestrofeminal, Equin, Estrace, Estrofem, Elleste Solo, Estring, Eastraderm TTS, Eastraderm Matrix, Dermestril, Premphase, Preempro, Prempak, Premique, Estratest, Estratest HS, Tibolone);

31) one or more of a modulator of transporters for noradrenaline, dopamine and/or serotonin, such as bupropion, GW-320659;

32) one or more of a purinergic receptor agonist and/or modulator;

33) one or more of a neurokinin (NK) receptor antagonist, including those described in WO-09964008;

34) one or more of an opioid receptor agonist, antagonist or modulator, preferably agonists for the ORL-1 receptor;

35) one or more of an agonist or modulator for oxytocin/vasopressin receptors, preferably a selective oxytocin agonist or modulator;

36) one or more modulators of cannabinoid receptors.

By cross reference herein to compounds contained in patents and patent applications which can be used in accordance with invention, we mean the therapeutically active compounds as defined in the claims (in particular of claim 1) and the specific examples (all of which is incorporated herein by reference).

If a combination of active agents is administered, then they may be administered simultaneously, separately or sequentially.

Auxiliary Agents—PDE5 Inhibitors

The suitability of any particular cGMP PDE5 inhibitor can be readily determined by evaluation of its potency and selectivity using literature methods followed by evaluation of its toxicity, absorption, metabolism, pharmacokinetics, etc in accordance with standard pharmaceutical practice.

IC50 values for the cGMP PDE5 inhibitors may be determined using the PDE5 assay (see hereinbelow).

Preferably the cGMP PDE5 inhibitors used in the pharmaceutical combinations according to the present invention are selective for the PDE5 enzyme. Preferably they are selective over PDE3, more preferably over PDE3 and PDE4. Preferably, the cGMP PDE5 inhibitors of the invention have a selectivity ratio greater than 100 more preferably greater than 300, over PDE3 and more preferably over PDE3 and PDE4.

Selectivity ratios may readily be determined by the skilled person. IC50 values for the PDE3 and PDE4 enzyme may be determined using established literature methodology, see S A Ballard et al, Journal of Urology, 1998, vol. 159, pages 2164-2171 and as detailed herein after.

Suitable cGMP PDE5 inhibitors for the use according to the present invention include:

the pyrazolo [4,3-d]pyrimidin-7-ones disclosed in EP-A-0463756; the pyrazolo [4,3-d]pyrimidin-7-ones disclosed in EP-A-0526004; the pyrazolo [4,3-d]pyrimidin-7-ones disclosed in published international patent application WO 93/06104; the isomeric pyrazolo [3,4-d]pyrimidin-4-ones disclosed in published international patent application WO 93/07149; the quinazolin-4-ones disclosed in published international patent application WO 93/12095; the pyrido [3,2-d]pyrimidin-4-ones disclosed in published international patent application WO 94/05661; the purin-6-ones disclosed in published international patent application WO 94/00453; the pyrazolo [4,3-d]pyrimidin-7-ones disclosed in published international patent application WO 98/49166; the pyrazolo [4,3-d]pyrimidin-7-ones disclosed in published international patent application WO 99/54333; the pyrazolo [4,3-d]pyrimidin-4-ones disclosed in EP-A-0995751; the pyrazolo [4,3-d]pyrimidin-7-ones disclosed in published international patent application WO 00/24745; the pyrazolo [4,3-d]pyrimidin-4-ones disclosed in EP-A-0995750; the compounds disclosed in published international application WO95/19978; the compounds disclosed in published international application WO 99/24433 and the compounds disclosed in published international application WO 93/07124. The pyrazolo [4,3-d]pyrimidin-7-ones disclosed in published international application WO 01/27112; the pyrazolo [4,3-d]pyrimidin-7-ones disclosed in published international application WO 01/27113; the compounds disclosed in EP-A-1092718 and the compounds disclosed in EP-A-1092719.

Further suitable PDE5 inhibitors for the use according to the present invention include:

5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756); 5-(2-ethoxy-5-morpholinoacetylphenyl)-1-methyl-3-n-propyl-1,6-dihydro-7H-pyrazolo[4,3-d]pyrimidin-7-one (see EP-A-0526004); 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166); 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333 ); (+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333); 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8); 5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15); 5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66); 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124); 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132); (6R, 12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8; 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433; and the compound of example 11 of published international application WO93/07124 (EISAI); and compounds 3 and 14 from Rotella D P, J. Med. Chem., 2000, 43,1257.

Still other suitable PDE5 inhibitors include:

4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; I-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

In vitro PDE inhibitory activities against cyclic guanosine 3′,5′-monophosphate (cGMP) and cyclic adenosine 3′,5′-monophosphate (cAMP) phosphodiesterases were determined by measurement of their IC₅₀ values (the concentration of compound required for 50% inhibition of enzyme activity).

The required PDE enzymes were isolated from a variety of sources, including human corpus cavernosum, human and rabbit platelets, human cardiac ventricle, human skeletal muscle and human and canine retina, essentially by the method of W. J. Thompson and M. M. Appleman (Biochem., 1971, 10, 311). In particular, the cGMP-specific PDE (PDE5) and the cGMP-inhibited cAMP PDE (PDE3) were obtained from human corpus cavernosum or human platelets; the cGMP-stimulated PDE (PDE2) was obtained from human corpus cavernosum and human platelets; the calcium/calmodulin (Ca/CAM)-dependent PDE (PDE1) from human cardiac ventricle; the cAMP-specific PDE (PDE4) from human skeletal muscle and human recombinant, expressed in SF9 cells; and the photoreceptor PDE (PDE6) from human or canine retina. Phosphodiesterases 7-11 were generated from full length human recombinant clones transfected into SF9 cells.

Assays can be performed either using a modification of the “batch” method of W. J. Thompson et al. (Biochem., 1979, 18, 5228) or using a scintillation proximity assay for the direct detection of AMP/GMP using a modification of the protocol described by Amersham plc under product code TRKQ7090/7100. In summary, the effect of PDE inhibitors was investigated by assaying a fixed amount of enzyme in the presence of varying inhibitor concentrations and low substrate, (cGMP or cAMP in a 3:1 ratio unlabelled to [³H]-labeled at a conc ˜⅓ Kₘ) such that IC₅₀≅Kᵢ. The final assay volume was made up to 100 μl with assay buffer [20 mM Tris-HCl pH 7.4, 5 mM MgCl₂, 1 mg/ml bovine serum albumin]. Reactions were initiated with enzyme, incubated for 30-60 min at 30° C. to give <30% substrate turnover and terminated with 50 μl yttrium silicate SPA beads (containing 3 mM of the respective unlabelled cyclic nucleotide for PDEs 9 and 11). Plates were re-sealed and shaken for 20 min, after which the beads were allowed to settle for 30 min in the dark and then counted on a TopCount plate reader (Packard, Meriden, Conn.) Radioactivity units were converted to % activity of an uninhibited control (100%), plotted against inhibitor concentration and inhibitor IC₅₀ values obtained using the ‘Fit Curve’ Microsoft Excel extension (or in-house equivalent). Results from these tests show that the compounds of the present invention are inhibitors of cGMP-specific PDE5.

Functional activity can be assessed in vitro by determining the capacity of a compound of the invention to enhance sodium nitroprusside or electrical field stimulation-induced relaxation of pre-contracted rabbit corpus cavernosum tissue strips, using methods based on that described by S. A. Ballard et al. (Brit. J. Pharmacol., 1996, 118 (suppl.), abstract 153P) or S. A. Ballard et al. (J. Urology, 1998,159, 2164-2171).

Compounds can be screened in vivo in anaesthetised dogs to determine their capacity, after i.v. administration, to enhance the pressure rises in the corpora cavernosa of the penis induced by intracavernosal injection of sodium nitroprusside, using a method based on that described by Trigo-Rocha et al. (Neurourol. and Urodyn., 1994, 13, 71).

Auxiliary Agents—NEP inhibitors (I:NEP)

NEP EC3.4.24.11 (FEBS Lett. 229(1), 206-210 (1988)), also known as enkephalinase or neprilysin, is a zinc-dependent neutral endopeptidase. This enzyme is involved in the breakdown of several bioactive oligopeptides, cleaving peptide bonds on the amino side of hydrophobic amino acid residues (Reviewed in Turner et al., 1997). The key neuronally released bioactive agents or neuropeptides metabolised by NEP include natriuretic peptides such as atrial natriuretic peptides (ANP) as well as brain natriuretic peptide and C-type natriuretic peptide, bombesin, bradykinin, calcitonin gene-related peptide, endothelins, enkephalins, neurotensin, substance P and vasoactive intestinal peptide. Some of these peptides have potent vasodilatory and neurohormone functions, diuretic and natriuretic activity or mediate behaviour effects. Background teachings on NEP have been presented by Victor A. McKusick et al on http://www3.ncbi.nlm.nih.gov/Omim/searchomim.htm.

The suitability of any particular I:NEP can be readily determined by evaluation of its potency and selectivity using literature methods followed by evaluation of its toxicity, absorption, metabolism, pharmacokinetics, etc in accordance with standard pharmaceutical practice.

Preferably the l:NEP have a selectivity over ACE of greater than 300.

IC50 values and selectivity ratios for ACE may be determined by methods described in EP1097719A1.

Examples of NEP inhibitors are disclosed and discussed in the following review articles: Pathol. Biol., 46(3), 1998, 191; Current Pharm. Design, 2(5), 1996, 443; Biochem. Soc. Trans., 21(3),1993, 678; Handbook Exp. Pharmacol., 104/1, 1993, 547; TiPS, 11, 1990, 245; Pharmacol. Rev., 45(1), 1993, 87; Curr. Opin. Inves. Drugs, 2(11), 1993, 1175; Antihypertens. Drugs, (1997), 113; Chemtracts, (1997), 10(11), 804; Zinc Metalloproteases Health Dis. (1996), 105; Cardiovasc. Drug Rev., (1996), 14(2), 166; Gen. Pharmacol., (1996), 27(4), 581; Cardiovasc. Drug Rev., (1994), 12(4), 271; Clin. Exp. Pharmacol. Physiol., (1995), 22(1), 63; Cardiovasc. Drug Rev., (1991), 9(3), 285; Exp. Opin. Ther. Patents (1996), 6(11), 1147.

Further examples of NEP inhibitors are disclosed in the following documents: EP-509442A; U.S. Pat. No. 192,435; U.S. Pat. No. 4,929,641; EP-599444B; U.S. Pat. No. 884,664; EP-544620A; U.S. Pat. No. 798,684; J. Med. Chem. 1993, 3821; Circulation 1993, 88(4), 1; EP-136883; JP-85136554; U.S. Pat. No. 4,722,810; Curr. Pharm. Design, 1996, 2, 443; EP-640594; J. Med. Chem. 1993, 36(1), 87; EP-738711-A; JP-270957; CAS # 115406-23-0; DE-19510566; DE-19638020; EP-830863; JP-98101565; EP-733642; WO9614293; JP-08245609; JP-96245609; WO9415908; JP05092948; WO-9309101; WO-9109840; EP-519738; EP-690070; J. Med. Chem. (1993), 36, 2420; JP-95157459; Bioorg. Med. Chem. Letts., 1996, 6(1), 65; EP-A-0274234; JP-88165353; Biochem.Biophys.Res. Comm.,1989, 164, 58; EP-629627-A; U.S. Pat. No. 77,978; Perspect. Med. Chem. (1993), 45; EP-358398-B

Further examples of NEP inhibitors are disclosed in EP1097719-A1, in particular compounds FXII to FXIII therein.

Preferred NEP inhibitors are compounds FV to FXI and F57 to F65 of EP1097719-A1.

Auxiliary Agents—NPY inhibitors (I:NPY)

The suitability of any particular l:NEP can be readily determined by evaluation of its potency and selectivity using literature methods followed by evaluation of its toxicity, absorption, metabolism, pharmacokinetics, etc in accordance with standard pharmaceutical practice.

An assay for identifying NPY inhibitors is presented in WO-A-98/52890 (see page 96, lines 2 to 28).

Bioavailability

Preferably, the compounds of the invention (and combinations) are orally bioavailable. Oral bioavailablity refers to the proportion of an orally administered drug that reaches the systemic circulation. The factors that determine oral bioavailability of a drug are dissolution, membrane permeability and metabolic stability. Typically, a screening cascade of firstly in vitro and then in vivo techniques is used to determine oral bioavailablity.

Dissolution, the solubilisation of the drug by the aqueous contents of the gastro-intestinal tract (GIT), can be predicted from in vitro solubility experiments conducted at appropriate pH to mimic the GIT. Preferably the compounds of the invention have a minimum solubility of 50 mcg/ml. Solubility can be determined by standard procedures known in the art such as described in Adv. Drug Deliv. Rev. 23, 3-25, 1997.

Membrane permeability refers to the passage of the compound through the cells of the GIT. Lipophilicity is a key property in predicting this and is defined by in vitro Log D_{7.4} measurements using organic solvents and buffer. Preferably the compounds of the invention have a Log D_{7.4} of −2 to +4, more preferably −1 to +2. The log D can be determined by standard procedures known in the art such as described in J. Pharm. Pharmacol. 1990, 42:144.

Cell monolayer assays such as CaCO₂ add substantially to prediction of favourable membrane permeability in the presence of efflux transporters such as p-glycoprotein, so-called caco-2 flux. Preferably, compounds of the invention have a caco-2 flux of greater than 2×10⁻⁶ cms⁻¹, more preferably greater than 5×10⁻⁶ cms⁻¹. The caco flux value can be determined by standard procedures known in the art such as described in J. Pharm. Sci, 1990, 79, 595-600.

Metabolic stability addresses the ability of the GIT or the liver to metabolise compounds during the absorption process: the first pass effect. Assay systems such as microsomes, hepatocytes etc are predictive of metabolic liability. Preferably the compounds of the Examples show metabolic stablity in the assay system that is commensurate with an hepatic extraction of less then 0.5. Examples of assay systems and data manipulation are described in Curr. Opin. Drug Disc. Devel., 201, 4, 36-44, Drug Met. Disp., 2000, 28, 1518-1523.

Because of the interplay of the above processes further support that a drug will be orally bioavailable in humans can be gained by in vivo experiments in animals. Absolute bioavailability is determined in these studies by administering the compound separately or in mixtures by the oral route. For absolute determinations (% absorbed) the intravenous route is also employed. Examples of the assessment of oral bioavailability in animals can be found in Drug Met. Disp., 2001, 29, 82-87; J. Med Chem, 1997, 40, 827-829, Drug Met. Disp., 1999, 27, 221-226.

Diagnostic Kits

The present invention also includes a diagnostic composition or diagnostic methods or kits for (i) detection and measurement of IK_{Ca} channel activity in biological fluids and tissue; and/or (ii) localization of a IK_{Ca} channel activity in erectile tissues; and/or for (iii) the detection of a predisposition to a SD, such as MED. In this respect, the composition or kit will comprise an entity that is capable of indicating the presence of one or more—or even the absence of one or more—targets, such as an IK_{Ca} channel targets in a test sample. Preferably, the test sample is obtained from male sexual genitalia or a secretion thereof or therefrom.

By way of example, the diagnostic composition may comprise any one of the nucleotide sequences mentioned herein or a variant, homologue, fragment or derivative thereof, or a sequence capable of hybridising to all or part of any one of the nucleotide sequence.

Probes

The diagnostic compositions and/or kits of the present invention may comprise probes such as nucleic acid hybridisation or PCR probes which are capable of detecting polynucleotide sequences, including genomic sequences, encoding a target coding region, such as an IK_{Ca} channel coding region or closely related molecules, such as alleles. The specificity of the probe, i.e., whether it is derived from a highly conserved, conserved or non-conserved region or domain, and the stringency of the hybridisation or amplification (high, intermediate or low) will determine whether the probe identifies only naturally occurring target coding sequence, or related sequences. Probes for the detection of related nucleic acid sequences are selected from conserved or highly conserved nucleotide regions of target family members and such probes may be used in a pool of degenerate probes. For the detection of identical nucleic acid sequences, or where maximum specificity is desired, nucleic acid probes are selected from the non-conserved nucleotide regions or unique regions of the target polynucleotides. As used herein, the term “non-conserved nucleotide region” refers to a nucleotide region that is unique to a target coding sequence disclosed herein and does not occur in related family members.

PCR as described in U.S. Pat. No. 4,683,195, U.S. Pat. No. 4,800,195 and U.S. Pat. No. 4,965,188 provides additional uses for oligonucleotides based upon target sequences. Such oligomers are generally chemically synthesized, but they may be generated enzymatically or produced from a recombinant source. Oligomers generally comprise two nucleotide sequences, one with sense orientation (5′→3′) and one with antisense (3′←5′) employed under optimised conditions for identification of a specific gene or condition. The same two oligomers, nested sets of oligomers, or even a degenerate pool of oligomers may be employed under less stringent conditions for detection and/or quantification of closely related DNA or RNA sequences.

The nucleic acid sequence for a target can also be used to generate hybridisation probes as previously described, for mapping the endogenous genomic sequence. The sequence may be mapped to a particular chromosome or to a specific region of the chromosome using well known techniques. These include in situ hybridisation to chromosomal spreads (Verma et al (1988) Human Chromosomes: A Manual of Basic Techniques, Pergamon Press, New York City), flow-sorted chromosomal preparations, or artificial chromosome constructions such as YACs, bacterial artificial chromosomes (BACs), bacterial PI constructions or single chromosome cDNA libraries.

In situ hybridisation of chromosomal preparations and physical mapping techniques such as linkage analysis using established chromosomal markers are invaluable in extending genetic maps. Examples of genetic maps can be found in Science (1995; 270:410f and 1994; 265:1981f). Often the placement of a gene on the chromosome of another mammalian species may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once a disease or syndrome has been crudely localised by genetic linkage to a particular genomic region any sequences mapping to that area may represent associated or regulatory genes for further investigation. The nucleotide sequence of the subject invention may also be used to detect differences in the chromosomal location due to translocation, inversion, etc. between normal, carrier or affected individuals.

Assay Methods

The diagnostic compositions and/or methods and/or kits may be used in the following techniques which include but are not limited to; competitive and non-competitive assays, radioimmunoassay, bioluminescence and chemiluminescence assays, fluorometric assays, sandwich assays, immunoradiometric assays, dot blots, enzyme linked assays including ELISA, microtiter plates, antibody coated strips or dipsticks for rapid monitoring of urine or blood, immunohistochemistry and immunocytochemistry. By way of example, an immunohistochemistry kit may also be used for localization of IK_{Ca} channel activity in erectile tissues and/or corpus cavernosal smooth muslce cells. This immunohistochemistry kit permits localization of a IK_{Ca} channel in tissue sections and cultured cells using both light and electron microscopy which may be used for both research and clinical purposes. Such information may be useful for diagnostic and possibly therapeutic purposes in the detection and/or prevention and/or treatment of a SD, such as MED. For each kit the range, sensitivity, precision, reliability, specificity and reproducibility of the assay are established. Intraassay and interassay variation is established at 20%, 50% and 80% points on the standard curves of displacement or activity.

Diagnostic Testing

In order to provide a basis for the diagnosis of disease, normal or standard values from a target should be established. This may be accomplished by combining body fluids or cell extracts taken from normal subjects, either animal or human, with, for example, an antibody to a target under conditions suitable for complex formation which are well known in the art. The amount of standard complex formation may be quantified by comparing it to a dilution series of positive controls where a known amount of antibody is combined with known concentrations of a purified target. Then, standard values obtained from normal samples may be compared with values obtained from samples from subjects potentially affected by a SD. Deviation between standard and subject values establishes the presence of the disease state.

A target itself, or any part thereof, may provide the basis for a diagnostic and/or a prophylactic and/or therapeutic compound. For diagnostic purposes, target polynucleotide sequences may be used to detect and quantify gene expression in conditions, disorders or diseases in which SD may be implicated.

The target encoding polynucleotide sequence may be used for the diagnosis of SD resulting from expression of the target. For example, polynucleotide sequences encoding a target may be used in hybridisation or PCR assays of tissues from biopsies or autopsies or biological fluids, to detect abnormalities in target expression. The form of such qualitative or quantitative methods may include Southern or northern analysis, dot blot or other membrane-based technologies; PCR technologies; dip stick, pin or chip technologies; and ELISA or other multiple sample formal technologies. All of these techniques are well known in the art and are in fact the basis of many commercially available diagnostic kits.

Such assays may be tailored to evaluate the efficacy of a particular therapeutic treatment regime and may be used in animal studies, in clinical trials, or in monitoring the treatment of an individual patient. In order to provide a basis for the diagnosis of disease, a normal or standard profile for target expression should be established. This is accomplished by combining body fluids or cell extracts taken from normal subjects, either animal or human, with the target or a portion thereof, under conditions suitable for hybridisation or amplification. Standard hybridisation may be quantified by comparing the values obtained for normal subjects with a dilution series of positive controls run in the same experiment where a known amount of purified target is used. Standard values obtained from normal samples may be compared with values obtained from samples from subjects potentially affected by a disorder or disease related to expression of the target coding sequence. Deviation between standard and subject values establishes the presence of the disease state. If disease is established, an existing therapeutic agent is administered, and treatment profile or values may be generated. Finally, the assay may be repeated on a regular basis to evaluate whether the values progress toward or return to the normal or standard pattern. Successive treatment profiles may be used to show the efficacy of treatment over a period of several days or several months.

Thus, in one aspect, the present invention relates to the use of a target polypeptide, or variant, homologue, fragment or derivative thereof, to produce anti-target antibodies which can, for example, be used diagnostically to detect and quantify target levels in SD states.

The present invention further provides diagnostic assays and kits for the detection of a target in cells and tissues comprising a purified target which may be used as a positive control, and anti-target antibodies. Such antibodies may be used in solution-based, membrane-based, or tissue-based technologies to detect any disease state or condition related to the expression of target protein or expression of deletions or a variant, homologue, fragment or derivative thereof.

The diagnostic compositions and/or kits comprising these entites may be used for a rapid, reliable, sensitive, and specific measurement and localization of an IK_{Ca} channel activity in erectile tissue extracts. In certain situations, the kit may indicate the existence a SD, such as MED.

Reporters

A wide variety of reporters may be used in the assay methods (as well as screens) of the present invention with preferred reporters providing conveniently detectable signals (eg. by spectroscopy). By way of example, a reporter gene may encode an enzyme which catalyses a reaction which alters light absorption properties.

Examples of reporter molecules include but are not limited to β-galactosidase, invertase, green fluorescent protein, luciferase, chloramphenicol, acetyltransferase, β-glucuronidase, exo-glucanase and glucoamylase. Alternatively, radiolabelled or fluorescent tag-labelled nucleotides can be incorporated into nascent transcripts which are then identified when bound to oligonucleotide probes.

In one preferred embodiment, the production of the reporter molecule is measured by the enzymatic activity of the reporter gene product, such as β-galactosidase.

A variety of protocols for detecting and measuring the expression of the target, such as by using either polyclonal or monoclonal antibodies specific for the protein, are known in the art. Examples include enzyme-linked immunosorbent assay (ELISA), radioimmunoassay (RIA) and fluorescent activated cell sorting (FACS). A two-site, monoclonal-based immunoassay utilising monoclonal antibodies reactive to two non-interfering epitopes on the target is preferred, but a competitive binding assay may be employed. These and other assays are described, among other places, in Hampton R et al (1990, Serological Methods, A Laboratory Manual, APS Press, St Paul Minn.) and Maddox Del. et al (1983, J Exp Med 15 8:121 1).

A wide variety of labels and conjugation techniques are known by those skilled in the art and can be used in various nucleic and amino acid assays. Means for producing labelled hybridisation or PCR probes for detecting the target polynucleotide sequences include oligolabelling, nick translation, end-labelling or PCR amplification using a labelled nucleotide. Alternatively, the target coding sequence, or any portion of it, may be cloned into a vector for the production of an mRNA probe. Such vectors are known in the art, are commercially available, and may be used to synthesize RNA probes in vitro by addition of an appropriate RNA polymerase such as T7, T3 or SP6 and labelled nucleotides.

A number of companies such as Pharmacia Biotech (Piscataway, N.J.), Promega (Madison, Wis.), and US Biochemical Corp (Cleveland, Ohio) supply commercial kits and protocols for these procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241. Also, recombinant immunoglobulins may be produced as shown in U.S. Pat. No. 4,816,567.

Additional methods to quantify the expression of a particular molecule include radiolabeling (Melby P C et al 1993 J Immunol Methods 159:235-44) or biotinylating (Duplaa C et al 1993 Anal Biochem 229-36) nucleotides, coamplification of a control nucleic acid, and standard curves onto which the experimental results are interpolated. Quantification of multiple samples may be speeded up by running the assay in an ELISA format where the oligomer of interest is presented in various dilutions and a spectrophotometric or calorimetric response gives rapid quantification.

Although the presence/absence of marker gene expression suggests that the gene of interest is also present, its presence and expression should be confirmed. For example, if the nucleotide sequence is inserted within a marker gene sequence, recombinant cells containing the same may be identified by the absence of marker gene function. Alternatively, a marker gene can be placed in tandem with a target coding sequence under the control of a single promoter. Expression of the marker gene in response to induction or selection usually indicates expression of the target as well.

Alternatively, host cells which contain the coding sequence for the target and express the target coding regions may be identified by a variety of procedures known to those of skill in the art. These procedures include, but are not limited to, DNA-DNA or DNA-RNA hybridisation and protein bioassay or immunoassay techniques which include membrane-based, solution-based, or chip-based technologies for the detection and/or quantification of the nucleic acid or protein.

Screens

Any one or more of appropriate targets—such as an amino acid sequence and/or nucleotide sequence—may be used for identifying an agent capable of modulating IK_{Ca} channel activity in any of a variety of drug screening techniques. The target employed in such a test may be free in solution, affixed to a solid support, borne on a cell surface, or located intracellularly. The abolition of target activity or the formation of binding complexes between the target and the agent being tested may be measured.

Techniques for drug screening may be based on the method described in Geysen, European Patent Application 84/03564, published on Sep. 13, 1984. In summary, large numbers of different small peptide test compounds are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in a drug screening techniques. Alternatively, non-neutralising antibodies can be used to capture the peptide and immobilise it on a solid support.

This invention also contemplates the use of competitive drug screening assays in which neutralising antibodies capable of binding a target specifically compete with a test compound for binding to a target.

Another technique for screening provides for high throughput screening (HTS) of agents having suitable binding affinity to the substances and is based upon the method described in detail in WO 84/03564.

It is expected that the assay methods of the present invention will be suitable for both small and large-scale screening of test compounds as well as in quantitative assays.

Thus, the present invention also relates to a method of identifying agents that mediate the relaxation of corpus cavernosum smooth muscle tone, the method comprising contacting a suitable target with the agent and then measuring the extent of relaxation of the relaxation of corpus cavernosum smooth muscle tone

The present invention also relates to a method of identifying agents that selectively modulating IK_{Ca} channel activity and/or mediate the relaxation of corpus cavernosum smooth muscle tone in sexual genitalia of an individual, the method comprising contacting a suitable target from the sexual genitalia of an individual and then measuring the IK_{Ca} channel activity and/or extent of relaxation of relaxation of corpus cavernosum smooth muscle tone.

The present invention also relates to a method of identifying agents that modulate the IK_{Ca} channel activity the method comprising contacting a suitable target with the agent and then measuring the activity and/or levels of expression of the IK_{Ca} channel.

The present invention also relates to a method of identifying agents that selectively modulate the IK_{Ca} channel activity the method comprising contacting a suitable target with the agent and then measuring the activity and/or levels of expression of the IK_{Ca} channel.

Animal Models

In vivo models may be used to investigate and/or design therapies or therapeutic agents to treat SDs, such as MED. The models could be used to investigate the effect of various tools/lead compounds on a variety of parameters which indicate the sexual arousal response.

The invention further provides transgenic nonhuman animals capable of expressing the nucleotide sequence encoding the IK_{Ca} channel of the present invention or a variant, homologue, derivative or fragment thereof and/or a transgenic nonhuman animal having one or more nucleotide sequence encoding the IK_{Ca} channel of the present invention or a variant, homologue, derivative or fragment thereof inactivated. Expression of such a nucleotide sequence is usually achieved by operably linking the nucleotide sequence to a promoter and optionally an enhancer, and microinjecting the construct into a zygote. See Hogan et al., “Manipulating the Mouse Embryo, A Laboratory Manual,” Cold Spring Harbor Laboratory. Inactivation of such a nucleotide sequence may be achieved by forming a transgene in which a cloned nucleotide sequence is inactivated by insertion of a positive selection marker. See Capecchi, Science 244, 1288-1292 (1989). The transgene is then introduced into an embryonic stem cell, where it undergoes homologous recombination with an endogenous variant gene. Mice and other rodents are preferred animals. Such animals provide screens and/or screening systems for identifying agents capable of modulating IK_{Ca} channel activity.

### EXAMPLES

The invention will now be further described only by way of example in which reference is made to the following Figures:

FIGURES

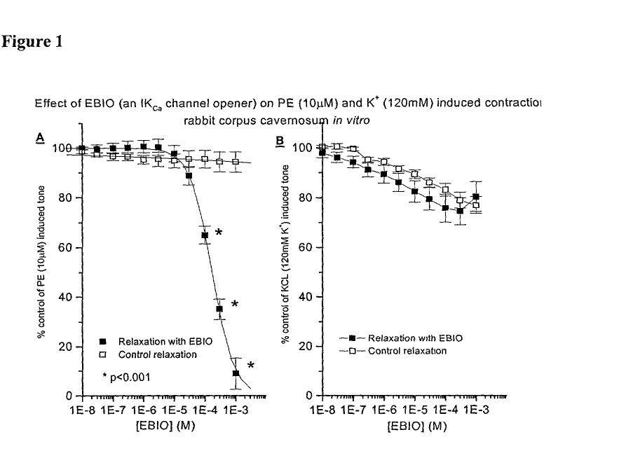

FIG. 1 which shows a graph;

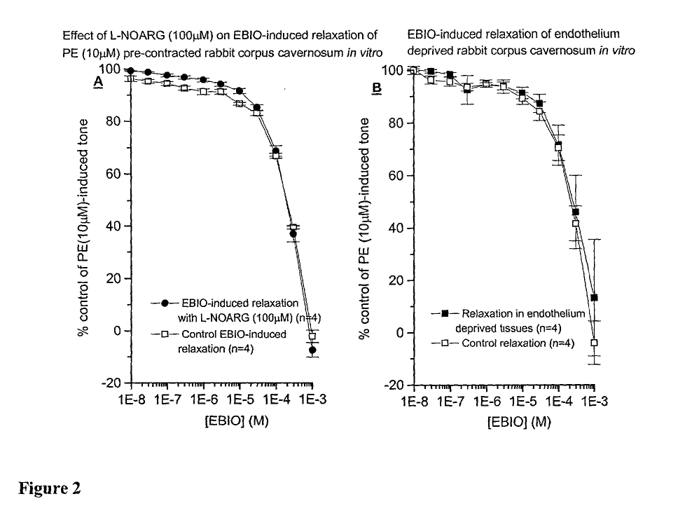

FIG. 2 which shows a graph;

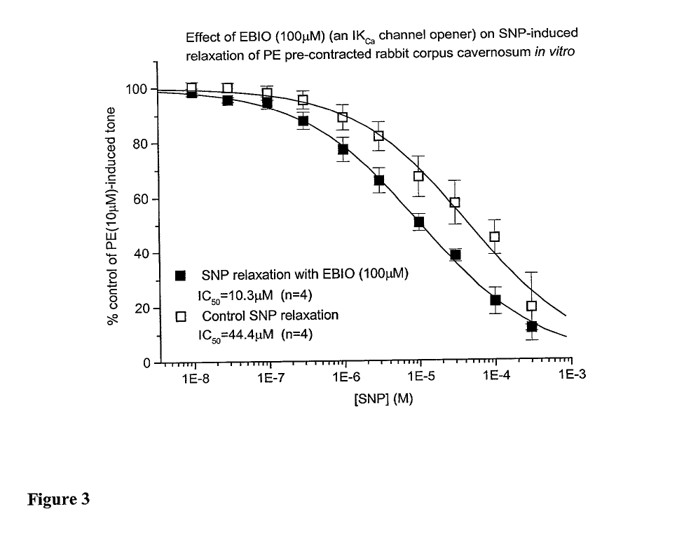

FIG. 3 which shows a graph;

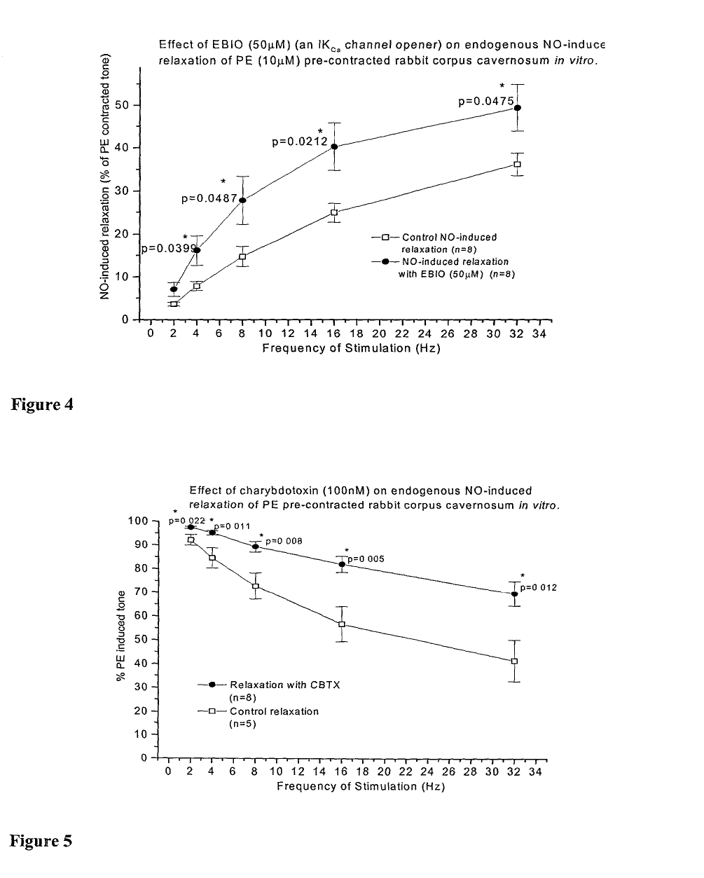

FIG. 4 which shows a graph;

FIG. 5 which shows a graph;

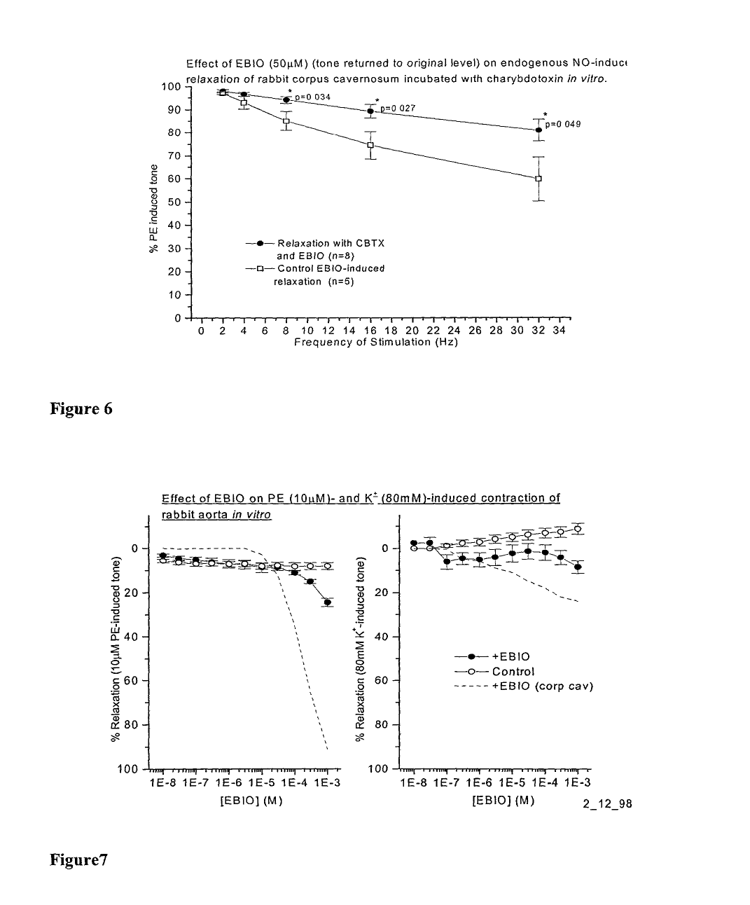

FIG. 6 which shows a graph;

FIG. 7 which shows a graph;

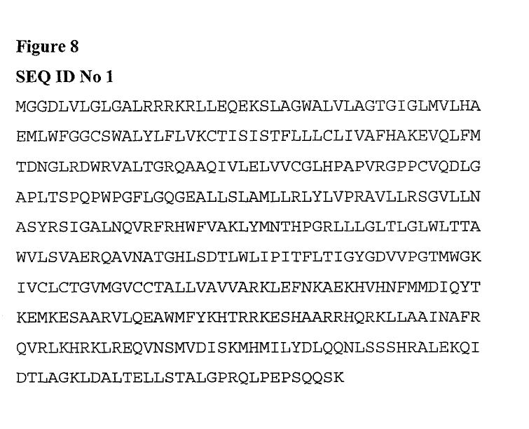

FIG. 8 which shows a sequence listing (SEQ ID No. 1); and

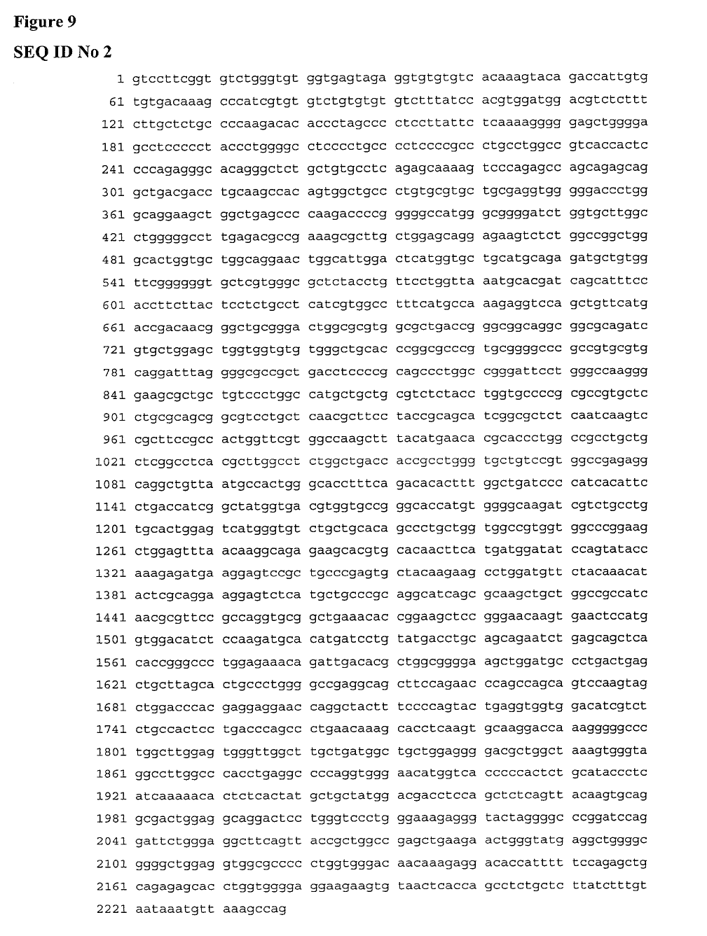

FIG. 9 which shows a sequence listing (SEQ ID No. 2).

In more detail:

FIG. 1 shows that the opening of IK_{Ca} channels, with EBIO (1-ethyl-2-benzimidazolinone, a commercially available channel opener from Aldrich Chem Co), induces a direct relaxation of corpus cavernosum. EBIO induces a concentration-dependent relaxation of PE-contracted rabbit corpus cavernosum in vitro, but has no effect in a high K⁺ environment. This suggests that EBIO relaxes the tissue via opening of IK_{Ca} channel and does not work by directly blocking VOCCs. Results are expressed as means±s.e. mean.

FIG. 2 shows that EBIO-induced relaxations of the corpus cavernosum are not via activation the NO/cGMP pathway or via endothelium dependent mechanism. EBIO-induced relaxation is still observed in the presence of a NOS inhibitor (L-NOARG) and in endothelium deprived rabbit corpus cavernosum in vitro. Results are expressed as means±s.e. mean.

FIG. 3 shows the opening of IK_{Ca} channels, with EBIO, potentiates SNP-induced relaxation of PE-contracted rabbit corpus cavernosum. EBIO (50 μM˜IC₂₀) potentiates SNP (0.1 nM-0.3 mM) induced relaxation of rabbit corpus cavernosum in vitro. Results are expressed as means±s.e. mean.

FIG. 4 shows the opening of IK_{Ca} channels, with EBIO, potentiates electrical field stimulated (EFS)-induced relaxation of PE-contracted rabbit corpus cavernosum. EBIO (50 μM) greatly potentiates EFS-induced relaxation of rabbit corpus cavernosum in vitro. EFS-induced relaxations were enhanced across the frequency range. Results are expressed as means±s.e. mean.

FIG. 5 shows the blockade of IK_{Ca} channels with charybdotoxin (ChTX—a toxin blocker of IK_{Ca} channels) reduces NO-mediated (EFS-induced) relaxations of the corpus cavernosum. ChTX (100 nM) significantly attenuates endogenous NO-induced relaxation by up to 30% of the corpus cavernosum in vitro. This significant reduction in EFS-induced relaxation occurs across the frequency range. Results are expressed as means±s.e. mean.

FIG. 6 shows the blockade of IK_{Ca} channels prevents EBIO-induced potentiation of nitrergic relaxations of the corpus cavernosum. The EBIO-induced potentiation of endogenous NO-mediated (EFS-induced) relaxation, observed in FIG. 4, is ChTX sensitive. 100 nM ChTX caused a reduction in control EBIO-induced relaxation of rabbit corpus cavernosum in vitro. Results are expressed as means±s.e. mean.

FIG. 7 shows the opening of IK_{Ca} channels has no effect on smooth muscle tone in the isolated rabbit aorta. EBIO has no relaxant effect on rabbit aorta in vitro at concentrations upto 100 μM. Thus suggesting that IK_{Ca} channels may not exist in some cardiovascular tissues. Results are expressed as means±s.e. mean.

FIG. 8 shows SEQ ID No. 1—details of which are as follows:

| | |
|---|---|
| LOCUS | NM_002250 2238 bp mRNA PRI 19-MAR-1999 |
| DEFINITION | Homo sapiens potassium intermediate/small conductance calcium- |
| | activated channel, subfamily N, member 4 (KCNN4) mRNA, and translated |
| | products. |
| ACCESSION | NM_002250 |
| VERSION | NM_002250.1 GI: 4504858 |
| SOURCE | human. |
| ORGANISM | Homo sapiens; Eukaryota; Metazoa; Chordata; Craniata; Vertebrata; |
| | Mammalia; Eutheria; Primates; Catarrhini; Hominidae; Homo. |
| REFERENCE | 1 (bases 1 to 2238) |
| AUTHORS | Logsdon, N. J., Kang, J., Togo, J. A., Christian, E. P. and Aiyar, J. |
| TITLE | A novel gene, hKCa4, encodes the calcium-activated |
| | potassium channel in human T lymphocytes |
| JOURNAL | J. Biol. Chem. 272 (52), 32723-32726 (1997) |
| MEDLINE | 98070459 |
| REFERENCE | 2 (bases 1 to 2238) |
| AUTHORS | Logsdon, N. J., Kang, J., Togo, J. A., Christian, E. P. and Aiyar, J. |
| TITLE | Direct Submission |
| JOURNAL | Submitted (04-SEP-1997) Target Discovery, Zeneca |
| | Pharmaceuticals, 1800 Concord Pike, Wilmington, DE 19897, USA |
| COMMENT REFSEQ: | This reference sequence was derived from |
| | AF022797. |
| PROVISIONAL | RefSeq: This is a provisional reference sequence |
| | record that has not yet been subject to human review. The final curated reference |
| | sequence record may be somewhat different from this one. |
| FEATURES | Location/Qualifiers |
| | source 1 . . . 2238 |
| | /organism=“Homo sapiens” |
| | /db_xref=“taxon:9606” |
| | /tissue_type=“lymph node” |
| | gene 1 . . . 2238 |
| | /gene=“KCNN4” |
| | /db_xref=“LocusID:3783” |
| | /db_xref=“MIM:602754” |
| CDS | 397 . . . 1680 |
| | /gene=“KCNN4” |
| | /codon_start=1 |
| | /db_xref=“LocusID:3783” |
| | /db_xref=“MIM:602754” |
| | /product=“potassium intermediate/small conductance |
| | calcium-activated channel, subfamily N, member 4” |
| | /protein_id=“NP_002241.1” |
| | /db_xref=“GI:4504859” |

FIG. 9 which shows SEQ ID No.2—details on which are as follows:

| | |
|---|---|
| polyA_signal | 2221..2226 |
| BASE COUNT | 421 a 666 c 707 g 444 t |

### Example 1

Materials and Methods

Isolation and Preparation of Tissues

Male New Zealand White rabbits (2.0-3.0 Kg) were killed by cervical dislocation. The abdominal cavity was opened and the penis excised, starting at the base of the organ at the pelvic bones. The corpus cavernosum was carefully dissected free from the surrounding tunica albuginea—2 tissue strips, approximately 10 mm×3 mm×2 mm in size, were obtained from each penis. The dissection was carried out in Krebs Ringer Solution. Tissues were used on the day of harvest.

Mounting Tissues in Organ Baths

The strips of corpus cavernosum and were mounted with surgical suture in 5 ml Wesley & Co. organ baths, and immersed in Kreb's solution maintained at 37° C. and aerated with 5% CO₂/95% O₂ to attain pH 7.4. The suture was connected to a force displacement transducer (Maywood Instruments Ltd.) and changes in isometric tension were recorded on the DART in vitro computer package. The tissues were put under an initial resting tension of 1.5 g and allowed to equilibrate for 1 hour. During equilibration, the tissues were rinsed at 5 ml/min. The approximate tissue tension after equilibration was 1 g. The tissues were then sensitised to contraction with phenylephrine (PE) (10 μM) and KCl (120 mM), and relaxation with sodium nitroprusside (SNP) (100 μM).

Direct Relaxant Effect of EBIO

Tissues were contracted with PE (10 μM) (≈EC₄₀) or 120 mM K⁺ and subjected to half-log unit cumulative additions of EBIO (10 nM-1 mM), with approximately 10 minute intervals between each addition.

Investigating the Mechanism of EBIO and Berberine-Induced Relaxation

1. Tissues were contracted with PE (10 μM) and incubated with N^{G}-nitro-I-arginine (L-NOARG—a NOS inhibitor) (300 μM) for 25 mins. The tissues were then subjected to cumulative half log unit additions of EBIO (10 nM-1 mM).

2. The endothelium lining of the lacunar spaces of rabbit corpus cavernosum was removed by mechanical disruption (rubbed gently between thumb and forefinger for 20-30s). Endothelium removal was checked by the degree of relaxation response to ACh (1 μM)—if the tissues did not relax, or relaxed poorly (<10% of maximal relaxation), they were considered to be functionally denuded of endothelium. Endothelium denuded tissues were contracted with PE (10 μM) and then subjected to an EBIO (10 nM-1 mM) does response curves.

Effect of EBIO on cGMP-mediated Relaxation

PE (10 μM) contracted tissues were subjected to half-log unit cumulative additions of SNP (cGMP mediated relaxations) (10 nM-300 μM) to obtain control dose response curves. After thorough washout (≈20 minutes at 5 ml/min), PE (10 μM) contracted tissues were incubated with EBIO (50 μM and 100 μM) for 15 minutes. Any drop in tone after addition of EBIO was returned to the pre-treatment level by further additions of PE, and then the tissues were subjected to SNP (10 nM-300 μM) dose response curves.

Effect of EBIO on Electrical Field Stimulation (EFS)-induced Relaxation

Tissues were bathed in “normal” Kreb's containing 5 μM guanethidine and 1 μM atropine, to block NA release and receptors respectively. EFS was delivered by a MS3 stimulator connected to 2 platinum electrodes which were placed at the top and bottom of the organ chambers. EFS parameters are as follows: Voltage=45-65V, Frequency=2-32 Hz, Pulse Width=0.2 ms, Train Duration=10 s. Tissues were contracted with PE (10 μM) and sensitised to EFS by stimulating at 8 Hz and 15 Hz and altering the voltage to obtain similar responses in all tissues. An EFS curve involved stimulating PE-contracted tissues at 2 Hz, 4 Hz, 8 Hz, 16 Hz and 32 Hz, and the degree of relaxation was measured by taking the minimum reading from the maximum reading obtained over a 30 s period. A control EFS response curve was always obtained, and then further EFS curves were performed after a 15 minute incubation with EBIO (50 μM and 100 μM) or charybdotoxin (100 nM; ChTX—a toxin blocker of IK_{Ca} channels), or both ChTX (100 nM) and EBIO (50 μM).

Selectivity of EBIO for Corpus Cavernosum over the Cardiovascular Tissue

PE (1 μM) or 80 mM K⁺ contracted rabbit aorta sections were subjected to half-log unit cumulative additions of EBIO (10 nM-1 mM) with approximately 10 minute intervals between each addition.

Drugs

Drugs used were: phenylephrine hydrochloride (PE), atropine sulphate, guanethidine, acetylcholine chloride (ACh), sodium nitroprusside (SNP), N^{G}-nitro-I-arginine (L-NOARG) (Calbiochem), KCl (Merck Ltd.), charybdotoxin (Tocris Cookson), 1-ethyl-2-benzimidazol-inone (EBIO) (Aldrich Chem. Co.). All drugs were dissolved in dH₂O except L-NOARG which was dissolved in Krebs, ChTX and EBIO which were dissolved in EtOH.

Kreb's Ringer Solution (Sigma Chemical Co.) was used, composition as follows: NaCl 118 mM, NaHCO₃ 25 mM, KCl 4.7 mM, KH₂PO₄ 1.2 mM, MgSO₄.7H₂O 1.2 mM, glucose 11 mM and CaCl₂ 2.5 mM. To prevent precipitation of reagents, the buffer solution was gassed (95% O₂/5% CO₂) for 10 minutes prior to adding the CaCl₂ solution.

Statistics

For each dose response curve, the relaxation responses are expressed as a percentage of the PE-induced contraction. The mean values±s.e. mean are then plotted against log[drug] concentration. Sigmoidal curves were fitted to the data using Origin curve fitting computer package. For the purpose of curve fitting, the minimum relaxation response is constrained to 0% and the maximum relaxation response is allowed to free fit. For EFS response curves, the degree of relaxation was expressed as a percentage of the PE-induced contraction. These values were then plotted against the frequency of stimulation.

Statistical analyses were made using the Student's paired t-test after a one-way analysis of variance.

Results

Opening IK_{Ca} Channels with EBIO Causes a Direct Relaxation of Corpus Cavernosum

EBIO suppresses basal tone (at concentrations≧10 μM) and induces concentration dependent relaxation of PE-contracted rabbit corpus cavernosum (FIG. 1). However, in a high K⁺ environment EBIO has no effect on contracted rabbit corpus cavernosum (FIG. 1). This suggests the mechanism of action of EBIO is via IK_{Ca} channel opening and not by directly blocking VOCCs.

EBIO-induced relaxations are not mediated via the NO/cGMP pathway or via an endothelium dependent mechanism

EBIO-induced relaxation is still observed in the presence of L-NOARG (100 μM) (a NOS inhibitor) (FIG. 2) and EBIO-induced relaxations are not affected by endothelium removal (FIG. 2).

Opening of IK_{Ca} Channels Potentiates Nitrergic Relaxant Mechanism

EBIO (50 μM˜IC₂₀) causes a potentiation of nitrergic-induced relaxation of rabbit corpus cavernosum (FIG. 3 and 4). EBIO enhanced both authentic endogenous nitrergic relaxations (EFS-induced) or exogenous cGMP-mediated relaxations (SNP-induced).

This enhancement was observed across the frequency/dose range of nitrergic relaxations (FIG. 3 and 4).

Evidence that IK_{Ca} channels are involved in nitrergic relaxations was determined using ChTX—a toxin blocker of IK_{Ca} channels. ChTX (100 nM) significantly attenuates endogenous NO-mediated (EFS-induced) relaxation of the corpus cavernosum by up to 30% (FIG. 5). This suggests that IK_{Ca} channels play a role in mediating nitrergic relaxations.

Since EBIO-induced potentiation of endogenous NO-induced relaxation is also ChTX (100 nM) sensitive (FIG. 6). This illustrates that the EBIO-induced potentiation of nitrergic relaxations is mediated by opening of IK_{Ca} channels.

IK_{Ca} Channel Opener—an Opportunity for Corpus Cavernosal Selectivity over the Cardiovascular System.

Tests with rabbit aorta in vitro have indicated that EBIO has no relaxant effect, except at high concentrations (≧100 μM) (FIG. 7). This suggests that IK_{Ca} channels openers may selectively relax corpus cavernosum without effecting the cardiovascular system.

Discussion

This study confirms that IK_{Ca} channels play an important functional role in the regulation of corpus cavernosal smooth muscle tone. The IK_{Ca} channel opener, EBIO, has concentration-dependent relaxant activity in isolated rabbit corpus cavernosum and can potentiate NO/cGMP-mediated relaxation. This relaxant effect is unaffected in endothelium deprived corpus cavernosum, indicating that the relaxant mechanism is endothelium-independent, and that IK_{Ca} channels are only present in the smooth muscle. The fact that L-NOARG (a NOS inhibitor) does not attenuate EBIO-induced relaxation also indicates that EBIO is not exerting its effect via the NO/cGMP pathway, and this is consistent with the endothelium-independent mechanism.

The relaxant activity of EBIO is strongly reduced in a high K⁺ environment. Since the increase in tonic tension obtained by depolarisation with K⁺ is due to the opening of calcium channels, the reduced effectiveness of EBIO in relaxing corpus cavernosum indicates that the drug does not directly block voltage operated calcium channels (VOCCs). This observation also confirms that EBIO is working via the K⁺ channel mechanism—the high extracellular K⁺ attenuates the K⁺ gradient across the plasma membrane, thus rendering the K⁺ channel-activating mechanism ineffective.

This study has also shown EBIO (50 μM) to potentiate NO-induced relaxation (SNP-induced) and endogenous NO-induced relaxation (EFS-induced). This study thus strengthens the proposal that IK_{Ca} channels may be modulated by NO, or PKG which is an effector of NO signalling. Charybdotoxin (ChTX—a toxin blocker of IK_{Ca} channels) significantly attenuates endogenous NO-induced relaxation suggesting that IK_{Ca} channels may have a role in setting the basal tone of this tissue. The EBIO-induced potentiation of endogenous NO-induced relaxation is also ChTX-sensitive, thus confirming that EBIO is working via IK_{Ca} channels. ChTX is not entirely selective for IK_{Ca} channels, and may also block BK_{Ca} channels. Therefore, these results cannot confirm that the EBIO-induced effects are attributable to IK_{Ca} channel modulation. Despite this, these results have clearly shown that K⁺ channel openers induce relaxation of corpus cavernosum and potentiate NO/cGMP-induced relaxation, and therefore represent a valid approach to treat SD, such as MED.

Summary

The present invention demonstrates for the first time that IK_{Ca} channels are expressed in corpus cavernosum smooth muscle cells. There are no literature reports to date relating to either IK_{Ca} channel expression in the corpus cavernosum. In addition, there are no literature reports which disclose any functional evidence for IK_{Ca} channels, such as penile IK_{Ca} channels in the smooth muscle cells of the corpus cavernosum. Any literature report relating to these channels describe a lack of expression throughout the cardiovascular system and/or the central nervous system (CNS).

The present invention demonstrates for the first time that smooth muscle relaxation via IK_{Ca} channel opening appears to be specific to the corpus cavernosum.

The present invention also demonstrates that IK_{Ca} channel openers, such as EBIO, are capable of enhancing a NO-induced relaxation of corpus cavernosum smooth muscle tone. This direct relaxation of corpus cavernosum smooth muscle tone is not endothelium dependent and does not result from either the inhibition of calcium channel activity or the direct stimulation of the NO pathway. This effect may also be observed in response to sexual arousal. Advantageously, EBIO has no effect on aortic smooth muscle tone and appear to show no effect on blood pressure in vivo.

The demonstration that modulation of IK_{Ca} channel activity mediates the relaxation of corpus cavernosal smooth muscle tone may be used to develop screens to identify agents capable of modulating IK_{Ca} channel activity. Such agents may be used to prevent and/or treat and/or enhance the erectile response and overcome an erectile dysfunction, such as a male erectile dysfunction (MED).

In one aspect, the present invention relates to a pharmaceutical composition for subsequent use in the treatment of a sexual dysfunction (SD); the pharmaceutical composition comprising an agent capable of modulating the activity of an intermediate conductance calcium-activated potassium (IK_{Ca}) channel in the sexual genitalia of an individual; wherein the agent is optionally admixed with a pharmaceutically acceptable carrier, diluent or excipient and wherein the modulation of the IK_{Ca} channel activity is capable of mediating a relaxation of corpus cavernosal smooth muscle tone.

In another aspect, the present invention relates to a pharmaceutical composition for subsequent use in the treatment of a sexual dysfunction (SD); the pharmaceutical composition comprising an agent capable of modulating the activity of an intermediate conductance calcium-activated potassium (IK_{Ca}) channel in the sexual genitalia of an individual; wherein the agent is optionally admixed with a pharmaceutically acceptable carrier, diluent or excipient and wherein the modulation of the IK_{Ca} channel activity is capable of mediating a relaxation of corpus cavernosal smooth muscle tone.

In another aspect, the present invention relates to the use of an agent in the preparation of a medicament for the treatment of a SD; wherein the agent is capable of modulating an IK_{Ca} channel activity in the sexual genitalia of an individual; wherein the agent is optionally admixed with a pharmaceutically acceptable carrier, diluent or excipient; and wherein the modulation of the IK_{Ca} channel activity is capable of mediating a relaxation of corpus cavernosal smooth muscle tone.

In another aspect, the present invention relates to the use of an agent in the preparation of a medicament for the treatment of a SD; wherein the agent is capable of modulating an IK_{Ca} channel activity in the sexual genitalia of an individual; wherein the agent is optionally admixed with a pharmaceutically acceptable carrier, diluent or excipient; and wherein the modulation of the IK_{Ca} channel activity is capable of mediating a relaxation of corpus cavernosal smooth muscle tone in response to sexual arousal.

In a further aspect, the present invention relates to a method for treating an individual; the method comprising delivering to the individual an agent that is capable of modulating IK_{Ca} channel activity in the sexual genitalia of the individual; wherein the agent is optionally admixed with a pharmaceutically acceptable carrier, diluent or excipient; and wherein the modulation of the IK_{Ca} channel activity is capable of mediating a relaxation of corpus cavernosal smooth muscle tone.

In a further aspect, the present invention relates to a method for treating an individual; the method comprising delivering to the individual an agent that is capable of modulating IK_{Ca} channel activity in the sexual genitalia of the individual; wherein the agent is optionally admixed with a pharmaceutically acceptable carrier, diluent or excipient; and wherein the modulation of the IK_{Ca} channel activity is capable of mediating a relaxation of corpus cavernosal smooth muscle tone in response to sexual arousal.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be covered by the present invention.

References

Alioua, A. et al (1998). The large conductance, voltage-dependent and calcium-sensitive K⁺ channel, Hslo, is a target of cGMP-dependent protein kinase phosphorylation in vivo. J. Biol. Chem. 273 (49): 32950-32956.

Berman J. R. et al (1999). Female sexual dysfunctioin: incidence, pathophysiology, evaluation and treatment options. Urology, 54: 385-391.

Benet, A. E. et al (1994), Male erectile dysfunction assessment and treatment options. Comp. Ther. 20: 669-673.

Boolel, M. et al (1996). Sildenafil, a novel effective oral therapy for male erectile dysfunction. Br. J. of Urology 78: 257-261.

Chiou, W. F. et al (1998). Relaxation of corpus cavernosum and raised intracavernous pressure by berberine in rabbit. Br. J. Pharmacol. 125: 1677-1684.

Christ, G. J. et al (1991). Intercellular communication through gap junctions: Potential role in pharmacomechanical coupling and syncytical tissue contraction in vascular smooth muscle isolated from the human corpus cavernosum. Life Sci. 49(24): PL195-200.

Christ, G. J. et al (1993). Characterisation of K currents in cultures human corporal smooth muscle cells. J. Androl. 14(5): 319-328.

Christ, G. J. et al (1995). Endothelin-1 as a putative modulator of erectile dysfunction: Characterisation of contraction of isolated corporal tissue strips. J. of Urology 153: 1998-2003.

Chuang, A. T. et al (1998). Sildenafil, a type-5 cGMP phosphodiesterase inhibitor, specifically amplifies endogenous cGMP-dependent relaxation in rabbit corpus

Fan, S. F. et al (1995). An analysis of the maxi-K⁺(K_{Ca}) channel in cultured human corporal smooth muscle cells. J. Urology 153: 818-825.

Fukao, M. et al (1999). Cyclic GMP-dependent protein kinase activates cloned BK_{Ca} channels expressed in mammalian cells by direct phosphorylation at serine 1072. J. Biol. Chem. 274(16): 10927-10935.

Ghanshani, S. et al. (1998). Human calcium-activated potassium channel gene KCNN4 maps to chromosome 19q13.2 in the region deleted in Diamond-Blackfan anemia. Genomics 51: 160-161, 1998.

Holmquist, F. et al (1990). K⁺-channel openers for relaxation of isolated penile erectile tissues from rabbit. J. Urology 144: 146-151.

Ishii, T. M. et al. (1997). A human intermediate conductance calcium-activated potassium channel. Proc. Nat. Acad. Sci. 94: 11651-11656.

Jeremy, J. Y. et al (1997). Effects of sildenafil, a type-5 cGMP phosphodiesterase inhibitor, and papaverine on cyclic GMP and cyclic AMP levels in the rabbit corpus cavernosum in vitro. Br. J. Urology 79: 958-963.

Joiner, W. J. et al (1997). hSK4, a member of a novel subfamily of calcium-activated potassium channels. Proc. Nat. Acad. Sci. 94: 11013-11018.

Leiblum, S. R. (1998). Definition and classification of female sexual disorders. Int J Impotence Res 10: S104-S106.

Lerner, S. E. et al (1993). A review of erectile dysfunction: new insights and more questions. J. Urology 149: 1246-1255.

Liu, Y. Z and Bridgeland, A. Unpublished results: (Pfizer, Sandwich; 1999).

Logsdon, N. J.; et al. (1997). A novel gene, hKCa4, encodes the calcium-activated potassium channel in human T lymphocytes. J. Biol. Chem. 272: 32723-32726.

Melman, A. & Gingell, J. C. (1999). The epidemiology and pathophysiology of erectile dysfunction. J. Urology 161: 5-11.

Montague, D. et al (1996). Clinical guidelines panel on erectile dysfunction: Summary report on the treatment of organic erectile dysfunction. J. Urology 156: 2007-2011.

Naylor, A. M. (1998). Endogenous neurotransmitters mediating penile erection. Br. J. Urology 81:424-431.

NIH Consensus Development Panel on Impotence (1993). NIH Consensus Conference Impotence. J.A.M.A. 270: 83.

Prieto, D., et al (1998). Contribution of K⁺ channels and ouabain-sensitive mechanisms to the endothelium-dependent relaxations of horse penile small arteries. Br. J. Pharmacol. 123, 1609-1620.

Quast, U. (1993). Do the K⁺ channel openers relax smooth muscle by opening K⁺ channels? TiPS 14: 332-336.

Simonsen, U., et al (1995). Involvement of nitric oxide in the non-adrenergic non-cholinergic neurotransmission of horse deep penile arteries: role of charybdotoxin-sensitive K⁺ channels. Br. J. Pharmacol. 116, 2582-2590.

Taub, H. C. et al (1993). Relationship between contraction and relaxation in human and rabbit corpus cavernosum. Urology 42(6): 698-704.

Abbreviations

BK_{Ca}: Large conductance calcium activated potassium channels (also referred to as Maxi K⁺ channels)

[Ca2]i: intracellular Ca²⁺ concentrations

sGC: soluble guanylate cyclase

cGMP: cyclic guanosine monophosphate

ChTX: Charybdotoxin: a toxin blocker of IK_{Ca} channels

EBIO: 1-ethyl-2-benzimidazolinone (a commercially available channel opener)

EFS: Electrical field stimulation which may be used to induce endogenous nitrergic mediated relaxation of corpus c smooth muscle tone.

IK_{Ca}: Intermediate conductance calcium activated potassium channels (also referred to as SK4 channels)

L-NOARG: An NOS inhibitor

NANC: non-adrenergic, non-cholinergic neurotransmission

NO mediated (EFS-induced)

NO: Nitric oxide: Synthesised from L-arginine by nitric oxide synthetase (NOS)

NOS: nitric oxide synthetase

PDE5: phosphodiesterase 5

PE: phenylephrine: induces contractions of smooth muscle

PKG: protein kinase G

SK_{Ca}: Small conductance calcium activated potassium channels

SNP: Sodium nitroprusside: induces endogenous cGMP-mediated relaxation of smooth muscle tone

VOCC: voltage activated calcium channels: activated by membrane depolarisation

1. A pharmaceutical composition for use (or when in use) in the treatment of a sexual dysfunction (SD); the pharmaceutical composition comprising an agent capable of modulating the activity of an intermediate conductance calcium-activated potassium (IK_{Ca}) channel in the sexual genitalia of an individual; wherein the agent is optionally admixed with a pharmaceutically acceptable carrier, diluent or excipient.

2. A pharmaceutical composition according to claim 1 wherein the modulation of the IK_{Ca} channel activity is capable of mediating a relaxation of corpus cavernosal smooth muscle tone.

3. A pharmaceutical composition according to claim 1 or claim 2 wherein the SD is a male SD (MSD).

4. A pharmaceutical composition according to claim 1 or 2 wherein the SD is an erectile dysfunction (ED).

5. A pharmaceutical composition according to claim 1 or 2 wherein the SD is a male erectile dysfunction (MED).

6. A pharmaceutical composition according to claim 1 or 2 wherein the composition is admixed with a pharmaceutically acceptable carrier, diluent or exciplent.

7. A method of treatment comprising administering to a subject an agent capable of modulating an IK_{Ca} channel activity in the sexual genitalia of said subject; wherein said agent is optionally admixed with a pharmaceutically acceptable carrier, diluent or excipient.

8. A method according to claim 7 wherein the modulation of the IK_{Ca} channel activity is capable of mediating a relaxation in corpus cavernosal smooth muscle tone.

9. A method according to claim 7 or 8 wherein said subject has a SD.

10. A method according to claim 9 wherein the SD is a male SD (MSD) or a female SD (FSD).

11. A method according to claim 10 wherein the SD is an erectile dysfunction (ED).

12. A method according to claim 9 wherein the SD is a male erectile dysfunction (MED).

13. A method according to claim 7 or 8 wherein the composition comprises a pharmaceutically acceptable carrier, diluent or excipient.

14. An assay method for identifying an agent capable of modulating an IK_{Ca} channel activity in order to treat a SD; the assay method comprising: contacting the agent with the IK_{Ca} channel; measuring the IK_{Ca} channel activity; wherein an increase in the IK_{Ca} channel activity is indicative that the agent may be useful in the treatment of the SD.

15. An assay method according to claim 15 wherein the SD is MED.

16. A process comprising the steps of:
(a) performing the assay according to claim 14 or claim 15;
(b) identifying one or more agents capable of modulating the IK_{Ca} channel activity; and
(c) preparing a quantity of those one or more identified agents.

17. A method of treating a SD with an agent; wherein the agent is capable of modulating an IK_{Ca} channel activity in an in vitro assay method; wherein the in vitro assay method is the assay method defined in claim 14 or claim 15.

18. Use of an agent in the preparation of a pharmaceutical composition for the treatment of a SD; wherein the agent is capable of modulating an IK_{Ca} channel activity when assayed in vitro by the assay method according to claim 14 or claim 15.

19. An agent identified by the assay method according to claim 14 or claim 15.

20. An agent according to claim 19 for use in medicine.

21. An agent according to claim 19 for use in treating a SD (preferably MED).

22. A medicament for oral administration to treat a SD (preferably MED); wherein the medicament comprises the agent according to claim 19.

23. A diagnostic method wherein the method comprises: isolating a sample from the sexual genitalia of an individual; determining whether the expression and/or IK_{Ca} channel activity in the sample from the individual has an effect on the relaxation of corpus cavernosal smooth muscle tone in the sexual genitalia of the individual.

24. A diagnostic composition or kit comprising means for detecting an entity in an isolated sample from the sexual genitalia of an individual; wherein the means can be used for determining whether the expression of the IK_{Ca} channel and/or the level of IK_{Ca} channel activity in the sample from the individual has an effect on the relaxation of corpus cavernosal smooth muscle tone in the sexual genitalia of the individual.

25. An animal model useful in the identification of agents capable of treating SD (in particular MED), said model comprising an anaesthetised animal including means to measure IK_{Ca} channel activity of the corpus cavernosal smooth muscle cells of said animal.

26. An assay method for identifying an agent capable of modulating IK_{Ca} channel activity in order to treat a SD (preferably MED); the assay method comprising: administering an agent to the animal model of claim 25; and measuring the IK_{Ca} channel open time probability in the sexual genitalia of said animal.

27. A method of identify agents capable of mediating the relaxation of corpus cavernosal smooth muscle tone comprising using an IK_{Ca} channel as a target.

28. A method according to claim 27 wherein the IK_{Ca} channel is used to screen for agents capable of modulating IK_{Ca} channel activity.

29. A method according to claim 28 wherein the modulation of the IK_{Ca} channel activity enhances nitrergic or nitric oxide-mediated relaxation of corpus cavernosal smooth muscle tone.

30. An IK_{Ca} channel and/or an agent as described in the accompanying Figures.

31. An IK_{Ca} channel and/or an agent substantially as described in the accompanying Figures for use in enhancing nitregic or nitric oxide-mediated relaxation of corpus cavernosal smooth muscle tone.


SEQUENCE LISTING (2 sequences)

SEQ ID NO: 1 - protein, 427 aa, Homo sapien
MGGDLVLGLGALRRRKRLLEQEKSLAGWALVLAGTGIGLMVLHAEMLWFGGCSWALYLFLVKCTISISTFLLLCLIVAFHAKEVQLFMTDNGLRDWRVALTGRQAAQIVLELVVCGLHPAPVRGPPCVQDLGAPLTSPQPWPGFLGQGEALLSLAMLLRLYLVPRAVLLRSGVLLNASYRSIGALNQVRFRHWFVAKLYMNTHPGRLLLGLTLGLWLTTAWVLSVAERQAVNATGHLSDTLWLIPITFLTIGYGDVVPGTMWGKIVCLCTGVMGVCCTALLVAVVARKLEFNKAEKHVHNFMMDIQYTKEMKESAARVLQEAWMFYKHTRRKESHAARRHQRKLLAAINAFRQVRLKHRKLREQVNSMVDISKMHMILYDLQQNLSSSHRALEKQIDTLAGKLDALTELLSTALGPRQLPEPSQQSK

SEQ ID NO: 2 - DNA, 2238 bp, Homo sapien
gtccttcggtgtctgggtgtggtgagtagaggtgtgtgtcacaaagtacagaccattgtgtgtgacaaagcccatcgtgtgtctgtgtgtgtctttatccacgtggatggacgtctctttcttgctctgccccaagacacaccctagcccctccttattctcaaaagggggagctggggagcctccccctaccctggggcctcccctgcccctccccgccctgcctggccgtcaccactccccagagggcacagggctctgctgtgcctcagagcaaaagtcccagagccagcagagcaggctgacgacctgcaagccacagtggctgccctgtgcgtgctgcgaggtgggggaccctgggcaggaagctggctgagccccaagaccccgggggccatgggcggggatctggtgcttggcctgggggccttgagacgccgaaagcgcttgctggagcaggagaagtctctggccggctgggcactggtgctggcaggaactggcattggactcatggtgctgcatgcagagatgctgtggttcggggggtgctcgtgggcgctctacctgttcctggttaaatgcacgatcagcatttccaccttcttactcctctgcctcatcgtggcctttcatgccaaagaggtccagctgttcatgaccgacaacgggctgcgggactggcgcgtggcgctgaccgggcggcaggcggcgcagatcgtgctggagctggtggtgtgtgggctgcacccggcgcccgtgcggggcccgccgtgcgtgcaggatttaggggcgccgctgacctccccgcagccctggccgggattcctgggccaaggggaagcgctgctgtccctggccatgctgctgcgtctctacctggtgccccgcgccgtgctcctgcgcagcggcgtcctgctcaacgcttcctaccgcagcatcggcgctctcaatcaagtccgcttccgccactggttcgtggccaagctttacatgaacacgcaccctggccgcctgctgctcggcctcacgcttggcctctggctgaccaccgcctgggtgctgtccgtggccgagaggcaggctgttaatgccactgggcacctttcagacacactttggctgatccccatcacattcctgaccatcggctatggtgacgtggtgccgggcaccatgtggggcaagatcgtctgcctgtgcactggagtcatgggtgtctgctgcacagccctgctggtggccgtggtggcccggaagctggagtttaacaaggcagagaagcacgtgcacaacttcatgatggatatccagtataccaaagagatgaaggagtccgctgcccgagtgctacaagaagcctggatgttctacaaacatactcgcaggaaggagtctcatgctgcccgcaggcatcagcgcaagctgctggccgccatcaacgcgttccgccaggtgcggctgaaacaccggaagctccgggaacaagtgaactccatggtggacatctccaagatgcacatgatcctgtatgacctgcagcagaatctgagcagctcacaccgggccctggagaaacagattgacacgctggcggggaagctggatgccctgactgagctgcttagcactgccctggggccgaggcagcttccagaacccagccagcagtccaagtagctggacccacgaggaggaaccaggctactttccccagtactgaggtggtggacatcgtctctgccactcctgacccagccctgaacaaagcacctcaagtgcaaggaccaaagggggccctggcttggagtgggttggcttgctgatggctgctggaggggacgctggctaaagtgggtaggccttggcccacctgaggccccaggtgggaacatggtcacccccactctgcataccctcatcaaaaacactctcactatgctgctatggacgacctccagctctcagttacaagtgcaggcgactggaggcaggactcctgggtccctgggaaagagggtactaggggcccggatccaggattctgggaggcttcagttaccgctggccgagctgaagaactgggtatgaggctggggcggggctggaggtggcgccccctggtgggacaacaaagaggacaccatttttccagagctgcagagagcacctggtggggaggaagaagtgtaactcaccagcctctgctcttatctttgtaataaatgttaaagccag